(19) United States
(12) Patent Application Publication
(10) Pub. No.: US 2020/0317749 A1
(43) Pub. Date: Oct. 8, 2020

(54) ALBUMIN VARIANTS FOR ENHANCED SERUM HALF-LIFE

(71) Applicant: DENALI THERAPEUTICS INC., South San Francisco, CA (US)

(72) Inventors: Mark S. Dennis, South San Francisco, CA (US); Mihalis Kariolis, South San Francisco, CA (US); Adam Silverman, South San Francisco, CA (US)

(73) Assignee: DENALI THERAPEUTICS INC., South San Francisco, CA (US)

(21) Appl. No.: 16/312,172

(22) PCT Filed: Jun. 29, 2017
(86) PCT No.: PCT/US2017/039857

§ 371 Date: Dec. 20, 2018

Related U.S. Application Data
(60) Provisional application No. 62/357,443, filed on Jul. 1, 2016.

(51) Int. Cl. C07K 14/765

(52) U.S. Cl. CPC C07K 14/765; A61K 38/00

(57) ABSTRACT

The present disclosure relates to albumin variants, derivatives and analogs thereof. In particular, provided are albumin variants that bind with increased efficiency to FcRn, including albumin variants that bind with increased efficiency at low pH levels but inefficiently or not at all at neutral pH levels. The albumin variants, derivatives, and analogs have an increased serum half-life when compared to naturally-occurring albumins.

### CROSS REFERENCE TO RELATED APPLICATIONS

This application claims the benefit of U.S. Provisional Application No. 62/357,443, filed Jul. 1, 2016, which is incorporated herein by reference in its entirety.

### FIELD

The present disclosure relates to albumin variants, derivatives and analogs thereof. In particular, the present disclosure provides albumin variants that bind with increased efficiency to FcRn, including albumin variants that bind with increased efficiency at low pH levels but inefficiently or not at all at neutral pH levels. The albumin variants, derivatives, and analogs have an increased serum half-life when compared to naturally-occurring albumins.

### BACKGROUND

Myeloid and endothelial cells internalize material from their local environment through bulk fluid-phase endocytosis. Most proteins within the resulting endosomes are directed to the lysosome for degradation; however, human serum albumin, as well as immunoglobulin G (IgG) proteins, can be rescued from this pathway through binding to the neonatal Fc receptor (FcRn). While albumin has negligible affinity to FcRn at neutral pH, it acquires an affinity in the low micromolar range to FcRn within the acidic environment of the endosome. Binding at low pH to FcRn protects albumin from lysosomal degradation while the absence of an appreciable affinity at neutral pH facilitates re-release into the plasma upon recycling back to the cell surface. This pH-dependent process of FcRn-mediated recycling results in albumin having an extended half-life of approximately 19 days in human serum.

Serum albumins represent approximately half of all serum protein in humans. Human serum albumin (HSA) is present in the serum at about 50 mg/ml and has a τ_{1/2} of about 19 days in humans. Albumin is present at extremely high concentrations in serum, accounting for roughly 50% of all protein. This abundance permits efficient recycling even within the context of its relatively weak native affinity for FcRn. FcRn binds near the interface of domains 1 and 3 on albumin. While residues from both domains participate in binding, the interaction is dominated by contributions from domain 3. High concentrations of serum albumin drive the binding reaction forward within the endosome.

The utility of albumin-fusion proteins as therapeutic agents with extended half-lives has been reported (e.g. Sleep, et al. Biochim Biophys Acta. 1830(12):5526 (2013)). Nonetheless, the extended in vivo half-life achieved by albumin fusions is typically significantly lower than unmodified native albumin. For example, Albiglutide, a fusion of two GLP-1 peptide repeats to albumin, has a half-life of 4-5 days in humans (Chen, et al. Exp Opin Drug Metab Toxicol. 8(5): 581 (2012)). The size and stability of the fusion partner is one contributing factor to this observation, but any modified, exogenously administered albumin fusion would constitute only a small fraction of total serum albumin, and thus would easily be outcompeted for FcRn binding and subsequent recycling. As a result, therapeutics looking to leverage albumin's desirable pharmacokinetics would benefit from improved FcRn binding properties. High-affinity albumin variants having modified domain 3 portions have reported a higher affinity for FcRn at both low and neutral pH. However, modifications in domain 3 that improve FcRn binding at low pH also concurrently enhance the affinity at neutral pH.

### SUMMARY

The present disclosure provides albumin proteins with optimal FcRn binding properties for enhanced FcRn-mediated recycling. For example, the albumin proteins include increased binding affinity for FcRn, and in some embodiments, the albumin proteins bind with increased affinity to FcRn in a pH-dependent manner. In some embodiments, the optimized albumin proteins bind very tightly to FcRn at low pH ranges (e.g. approximately pH 5.0-6.5) and very weakly or not at all to FcRn at neutral to higher pH ranges (e.g. approximately pH 7.0-7.5). The present disclosure further provides enhanced albumin polypeptides that preferentially bind to FcRn in acidic early endosomes and are released to the serum once the endosome recycles to the cell surface.

Thus, the present disclosure provides an albumin variant polypeptide, comprising at least one amino acid substitution in serum albumin domain 1, and wherein the at least one amino acid substitution enhances the specific binding between the albumin variant polypeptide and an FcRn polypeptide. Another aspect of the present disclosure includes an albumin variant polypeptide, comprising at least one amino acid substitution in a structural region that does not directly interact with an FcRn polypeptide, wherein the at least one amino acid substitution enhances the specific binding between the albumin variant polypeptide and the FcRn polypeptide.

Thus, an aspect includes an albumin variant polypeptide, comprising at least one amino acid substitution in SEQ ID NO:2, wherein said substitution is at a position selected from the group consisting of 1, 3, 5, 6, 7, 10, 11, 12, 13, 14, 15, 16, 18, 19, 20, 24, 25, 27, 30, 31, 32, 34, 37, 40, 41, 43, 44, 50, 51, 56, 57, 60, 61, 64, 67, 68, 76, 77, 78, 87, 88, 89, 90, 92, 93, 94, 95, 99, 101, 106, 109, 111, 112, 116, 119, 120, 130, 136, 137, 138, 142, 145, 149, 152, 153, 156, 157, 159, 160, 162, 163, 165, 170, 171, 174, 183, 184, 188, 189, 190, 191, 192, 193, 194, 196, 198, and 199, and wherein said albumin variant polypeptide specifically binds FcRn. In certain embodiments, the binding is with higher affinity than the corresponding unmodified albumin polypeptide.

Certain embodiments include an albumin variant polypeptide comprising at least one amino acid substitution selected from the group consisting of D1G, H3Y, S5P, R10G, K12R, D13G, N18D, K20E, I25V, F27L, F27S, C34R, A50V, K51R, E60G, K64E, T76A, V77I, A78T, C90W, K93E, E95G, C101W, K106R, N111D, V116A, E119G, V120A, N130D, K136E, K137R, I142V, F149S, E153G, F156S, F157L, K159R, R160G, K162R, K162E, F165L, Q170R, K174R, D183G, E184G, E188G, K190E, S192P, L198P, and K199R

Certain embodiments include an albumin variant polypeptide comprising at least one amino acid substitution selected from the group consisting of D1G, S5P, E6G, V7A, R10G, F11S, K12R, D13G, L14P, G15D, E16G, N18D, F19L, K20E, K20R, L24S, I25V, F27C, F27L, Y30H, Q32R, E37K, V40A, K41E, V43I, N44E, A50V, K51R, D56G, D56T, E57G, N61S, K64E, T76A, A78T, A88V, A92T, K93E, Q94R, E95G, N99S, K106R, N109D, N111D, N111E, E119G, N130D, K136E, Y138H, I142V, I142T, R145G, P152S, F156S, K162R, K162E, A163T, A171V, E184G, E188G, G189R, K190E, A191V, S192P, A194T, Q196R, L198P, K199R, and K199E.

Certain embodiments include an albumin variant polypeptide comprising at least one amino acid substitution selected from the group consisting of S5P, D13G, N18D, K20E, L24S, K41E, K41R, V43A, D56G, H67Y, T68A, M87I, K93E, E95G, N99D, K106R, N111S, L112S, I142T, F149S, P152S, P152L, R160G, F165L, K190E, K190R, L198P, K199R, and K199E.

Certain other embodiments include an albumin variant polypeptide comprising at least one amino acid substitution selected from the group consisting of Y30C, L31S, Q32R, K41E, A50T, K51R, T76A, D89G, N130D, K162E, E188G, K190E, and S192P. Specific embodiments include an albumin variant polypeptide comprising any one of or a plurality of the amino acid substitutions disclosed herein. Other specific embodiments include albumin variant polypeptides comprising the amino acid substitution F19L, L24S, A88V, K93E, F149S, K190E, S192P, Q196R, or K199E. In certain embodiments, the albumin variant polypeptide is a human serum albumin.

In some embodiments, the albumin variant polypeptide has a sequence set forth in any one of SEQ ID NOS:9-16. In other embodiments, the albumin variant of polypeptide has any one or more of the amino acid variations found in SEQ ID NOS:9-17 when compared to SEQ ID NO:2. In other embodiments, the albumin variant polypeptide is derived from a human albumin.

Another aspect includes an albumin variant polypeptide that specifically binds to an FcRn protein (e.g. the protein having the sequence of SEQ ID NO:5) with increased affinity compared to the serum albumin of SEQ ID NO:2. IN another aspect, the albumin variant polypeptide specifically binds to an FcRn protein with increased affinity at a pH of about 5.5 when compared to the serum albumin of SEQ ID NO:2. In certain embodiments, the albumin variant polypeptides bind to an FcRn protein with increased affinity at an acidic pH when compared to its affinity at a neutral pH. In other preferred embodiments, the albumin variant polypeptide specifically binds to an FcRn protein with an increased affinity at a pH of about 5.5 when compared to its affinity at a pH of about 7.4.

In certain specific embodiments, the albumin variant polypeptides comprise a first binding affinity for said FcRn at a pH of about 5.5 and a second binding affinity for said FcRn at a pH of about 7.4, wherein said first binding affinity is between 10 and 20 fold, between 21 and 30 fold, between 31 and 40 fold, between 41 and 50 fold, between 51 and 60 fold, between 61 and 70 fold, between 71 and 80 fold, between 81 and 90 fold, between 91 and 100 fold, between 101 and 1,000 fold, or between 10,001 and 100,000 fold than said second binding affinity.

In specific embodiments, the albumin variant polypeptide comprises a binding affinity for said FcRn at a pH of about 5.5 and does not exhibit any detectable specific binding for said FcRn at pH 7.4 (e.g. when measured by ELISA, Biacore, or other standard methods known in the art for detecting protein-protein interactions). In other specific embodiments, the albumin variant polypeptides of the present disclosure specifically binds to an FcRn protein at a pH of about 5.5 and does not specifically bind to said FcRn at a pH of about 7.4.

In other embodiments, the albumin variant polypeptides bind to an FcRn protein with a Kd of less than about 104, less than about 100 nM, or less than about 10 nM at a pH of about 5.5. In other embodiments, the albumin variant polypeptides bind to an FcRn protein with a Kd of more than about 1 μM, less than about 1 μM, or less than about 100 nM at a pH of about 7.4.

In other embodiments the albumin variant polypeptide specifically binds to an FcRn protein with a Kd of less than about 1 μM, less than about 100 nM, or less than about 10 nM at a pH of about 5.5. In other embodiments, the albumin variant polypeptide specifically binds to an FcRn protein with a Kd of more than about 1 μM, less than about 1 μM, or less than about 100 nM at a pH of about 7.4.

Another aspect includes albumin variant polypeptides as described herein further comprising one or more additional amino acid modifications.

Another aspect includes an albumin variant polypeptide, wherein at least a portion of said polypeptide comprises an amino acid sequence having at least a 90% sequence identity to SEQ ID NO:2 and wherein said albumin variant polypeptide specifically binds to FcRn at a pH of about 5.5. The present disclosure further provides albumin variant polypeptides, comprising amino acid sequences having at least a 90% sequence identity to amino acid numbers 1-199 of SEQ ID NO:2 and wherein said albumin variant polypeptide specifically binds to FcRn at a pH of about 5.5. In certain embodiments, the albumin variant polypeptide specifically binds to FcRn at a pH of about 5.5 with increased affinity compared to the corresponding unmodified albumin, for example, HSA.

The present disclosure provides an albumin variant polypeptide, wherein at least a portion of the polypeptide comprises an amino acid sequence having at least a 90% sequence identity to SEQ ID NO:9, SEQ ID NO:10, SEQ ID NO:11, SEQ ID NO:12, SEQ ID NO:13, SEQ ID NO:14, SEQ ID NO:15, SEQ ID NO:16, or SEQ ID NO:17 and wherein said albumin variant polypeptide specifically binds to FcRn at a pH of about 5.5.

Another aspect includes a nucleic acid molecule encoding an albumin variant polypeptide disclosed herein. In some embodiments, the nucleic acid molecules comprise deoxyribonucleotides. In other embodiments, the nucleic acid molecules comprise ribonucleotides.

Another aspect includes a vector operable to express an albumin variant polypeptide described herein. In certain embodiments, a vector comprises bacterial, bacteriophage, fungal, viral, insect, or mammalian expression control sequences.

Another aspect includes a cell comprising the nucleic acid molecules described herein, wherein an albumin variant polypeptides is expressed from the vectors described herein.

Another aspect includes a pharmaceutical composition, comprising an albumin variant polypeptide disclosed herein. Certain embodiments include medicaments comprising the albumin variant polypeptides disclosed herein.

Another aspect includes a pharmaceutical composition, comprising a nucleic acid molecule disclosed herein. Certain embodiments include medicaments comprising the nucleic acid molecules disclosed herein.

Another aspect includes a use of an albumin variant polypeptide disclosed herein for therapy.

Another aspect includes methods of treating a disease, comprising administering an effective amount of a composition comprising an albumin variant polypeptide disclosed herein to a patient in need thereof.

Another aspect includes a method of manufacturing an albumin variant polypeptide disclosed herein, comprising transferring a nucleic acid molecule operable to express an albumin variant polypeptide into an expression system and expressing said albumin variant polypeptide from said nucleic acid molecule. An embodiment includes the further step of recovering the albumin variant from the expression system.

Another aspect includes a method of manufacturing an albumin variant polypeptide disclosed herein, comprising synthesizing a polypeptide in an in vitro synthesis reaction. In an example, the in vitro synthesis reaction is selected from the group consisting of cell-free protein synthesis, liquid phase protein synthesis, and solid phase protein synthesis.

### BRIEF DESCRIPTION OF THE DRAWINGS

FIG. 1 shows a schematic for selecting albumin variants using a yeast surface display approach. Albumin was fused with the C-terminus of the yeast cell wall protein Aga2p. A chicken polyclonal anti-C-myc antibody was bound to the albumin/C-myc fusions at the cell surface and visualized with Alexa Fluor® 488 conjugated goat anti-chicken polyclonal antibodies. Binding was simultaneously assessed using biotinylated FcRn alpha chain extracellular domain/β₂ microglobulin heterodimers and fluorescently-labeled streptavidin (SAV, denoted by a diamond). Amino acid Nos. 24-297 of the FcRn alpha subunit (SEQ ID NO:5) and amino acid Nos. 21-119 of the β₂ microglobulin (SEQ ID NO:18) were used.

FIG. 2 shows a Fluorescence Activated Cell Sorter (FACS) plot for pH 7.4 sort (sort 6) of the shuffled library. The x-axis shows albumin expression and display on the surface of yeast cells and the y-axis shows FcRn binding to the albumin at pH 7.4. The three windows identify the high, moderate, and negative pools for pH 7.4

FIG. 3A shows pH-dependent binding of 1000 nM FcRn to single yeast clones displaying the albumin variants isolated from Sort 7 or Sort 6high.

FIG. 3B shows pH-dependent binding of 4 nM FcRn to single yeast clones displaying the albumin variants isolated from Sort 7 or Sort 6high.

### DETAILED DESCRIPTION

The present disclosure relates to albumin variants, derivatives and analogs thereof. An aspect includes an albumin variant that specifically binds with increased efficiency to FcRn at low pH levels but inefficiently or not at all at neutral pH levels. The albumin variants, derivatives, and analogs include an increased serum half-life when compared to naturally-occurring albumins, for example HSA.

An aspect includes an albumin variant protein with an improved FcRn binding profile with engineered Domain 1 portions. FcRn is a heterodimer of a non-polymorphic MHC class I-like alpha chain (SEQ ID NO:5) and β₂ microglobulin (SEQ ID NO:18). Domain 1 shall be defined herein as amino acid numbers 1-199 of SEQ ID NO:2 or as SEQ ID NO:6. Domain 1 contributes a small number of residues that interact directly with FcRn but contains numerous loops and helices that provide structural stability for the interaction. Additionally, domain 1 undergoes significant structural rearrangements with respect to domain 3 upon FcRn binding. Accordingly, variants providing additional stability or rigidity within these positions enhance the albumin-FcRn binding even though they are not directly involved in the binding interaction. In an example, albumin variant polypeptides were prepared using error-prone PCR by, for example, randomly incorporating mutations throughout the nucleic acids that encode albumin variant proteins. This resulted in altered residues that affect binding to FcRn.

In describing and claiming one or more embodiments of the present disclosure, the following terminology will be used in accordance with the definitions described below:

The singular form “a”, “an”, and “the” includes plural references unless indicated otherwise.

The term “absorption” is the movement of a drug into the bloodstream. A drug needs to be introduced via some route of administration. For example, drugs of provided herein may be delivered by oral, buccal, topical, dermal, inhalation, nasal, subcutaneous, intramuscular, or intravenous route or by any other route known in the pharmaceutical arts. Exemplary dosage forms include a solution, emulsion, inhalable powder, suspension, tablet, patch, capsule or other liquid.

An “albumin variant” may variously be referred to as “derivatives,” “analogs,” modified serum albumin polypeptides (MSA), or portions thereof. They include at least one amino acid modification, such as a deletion, substitution, addition, or a set of amino acid modifications, that affect albumin binding to FcRn. The variations may localize at any position of the wild type serum albumin or to a portion or a fragment thereof. In some embodiments, there may be 1, 2, 3, 4, 5, 6, 7, 8, 9, or 10 amino acid changes within domain 1, or multiple changes to the domains in any combination as disclosed herein. In certain embodiments, an MSA polypeptide that is a variant of a fragment of a wild type serum albumin has a minimal length required for binding to FcRn. In some embodiments, an albumin variant polypeptide comprises a variant domain region. In certain embodiments, such variant domain region retains a similar three-dimensional fold of the corresponding domain of unmodified or wild-type serum albumin. Mature, wild-type human serum albumin is a 585 amino acid polypeptide that has the sequence of SEQ ID NO:2.

“Binding affinity” generally refers to the strength of the sum total of the noncovalent interactions between an albumin variant and FcRn. Unless indicated otherwise, as used herein, “binding affinity” refers to intrinsic binding affinity which reflects a 1:1 interaction. The affinity of these molecules for each other may generally be represented by the equilibrium dissociation constant (Kd or K_{D}), which is calculated as the ratio k_{off}/kₒₙ. See, e.g., Chen, Y, et al., (1999) J. Mol. Biol 293:865-881. Affinity can be measured by known methods such as BIAcore and surface plasmon resonance (SPR) assays. By way of example, binding or affinity (Ka and/or Kd) may be evaluated in vitro using, for example, any one or more of the assays described in the examples or other binding assays such as SPR assays. Similarly, k_{off} and/or kₒₙ may be evaluated in vitro using, for example, any one or more of the assays described in the examples or other binding assays, for example SPR assays.

A “clinician” or “medical researcher” or “veterinarian” as used herein, can include, without limitation, doctors, nurses, physician assistants, lab technicians, research scientists, clerical workers employed by the same, or any person involved in determining, diagnosing, aiding in the diagnosis or influencing the course of treatment for the individual.

An “effective amount” refers to an amount of therapeutic compound that is effective, at dosages and for periods of time necessary, to achieve the desired therapeutic or prophylactic result. A “therapeutically effective amount” of a therapeutic compound may vary according to factors such as the disease state, age, sex, and weight of the individual. A therapeutically effective amount may be measured, for example, by improved survival rate, more rapid recovery, or amelioration, improvement or elimination of symptoms, or other acceptable biomarkers or surrogate markers. A “therapeutically effective amount” is also one in which any toxic or detrimental effects of the therapeutic compound are outweighed by the therapeutically beneficial effects. A “prophylactically effective amount” refers to an amount of therapeutic compound that is effective, at dosages and for periods of time necessary, to achieve the desired prophylactic result. Typically, but not necessarily, since a prophylactic dose is used in subjects prior to or at an earlier stage of disease, the prophylactically effective amount will be less than the therapeutically effective amount.

“Homologs” are bioactive molecules that are similar to a reference molecule at the nucleotide sequence, peptide sequence, functional, or structural level. Homologs may include sequence derivatives that share a certain percent identity with the reference sequence. Thus, in one embodiment, homologous or derivative sequences share at least a 70 percent sequence identity. In a specific embodiment, homologous or derivative sequences share at least an 80 or 85 percent sequence identity. In a specific embodiment, homologous or derivative sequences share at least a 90 percent sequence identity. In a specific embodiment, homologous or derivative sequences share at least a 95 percent sequence identity. In a more specific embodiment, homologous or derivative sequences share at least a 50, 55, 60, 65, 70, 75, 85, 86, 87, 88, 89, 90, 91, 92, 93, 94, 95, 96, 97, 98, or 99 percent sequence identity. Homologous or derivative nucleic acid sequences may also be defined by their ability to remain bound to a reference nucleic acid sequence under high stringency hybridization conditions. Homologs having a structural or functional similarity to a reference molecule may be chemical derivatives of the reference molecule. Methods of detecting, generating, and screening for structural and functional homologs as well as derivatives are known in the art.

“Hybridization” generally depends on the ability of denatured DNA to reanneal when complementary strands are present in an environment below their melting temperature. The higher the degree of desired homology between the probe and hybridizable sequence, the higher the relative temperature that can be used. As a result, it follows that higher relative temperatures would tend to make the reaction conditions more stringent, while lower temperatures less so. For additional details and explanation of stringency of hybridization reactions, see Ausubel et al, Current Protocols in Molecular Biology, Wiley Interscience Publishers (1995).

The term percent “identity,” in the context of two or more nucleic acid or polypeptide sequences, refer to two or more sequences or subsequences that have a specified percentage of nucleotides or amino acid residues that are the same, when compared and aligned for maximum correspondence, as measured using one of the sequence comparison algorithms described below (e.g., BLASTP and BLASTN or other algorithms available to persons of skill) or by visual inspection. Depending on the application, the percent “identity” can exist over a region of the sequence being compared, e.g., over a functional domain, or, alternatively, exist over the full length of the two sequences to be compared. For sequence comparison, typically one sequence acts as a reference sequence to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters.

Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by visual inspection (see generally Ausubel et al., infra).

One example of an algorithm that is suitable for determining percent sequence identity and sequence similarity is the BLAST algorithm, which is described in Altschul et al., J. Mol. Biol. 215:403-410 (1990). Software for performing BLAST analyses is publicly available through the National Center for Biotechnology Information (www.ncbi.nlm.nih.gov/).

An “individual,” “subject” or “patient” is a vertebrate. In certain embodiments, the vertebrate is a mammal. Mammals include, but are not limited to, primates (including human and non-human primates), rodents (e.g., mice, hamsters, guinea pigs, and rats), farm animals, sport animals, and pets (e.g. dogs and cats). In certain embodiments, a mammal is a human.

“LNA” or “locked nucleic acid” or “inaccessible RNA” is a modified RNA nucleotide. The ribose moiety of an LNA nucleotide is modified with an extra bridge connecting the 2′ oxygen and 4′ carbon. The bridge “locks” the ribose in the 3′-endo (North) conformation, which is often found in the A-form duplexes. LNA nucleotides can be mixed with DNA or RNA residues in the oligonucleotide whenever desired and hybridize with DNA or RNA.

A “medicament” is an active drug that has been manufactured for the treatment of a disease, disorder, or condition.

“Morpholinos” are synthetic molecules that are non-natural variants of natural nucleic acids that utilize a phosphorodiamidate linkage.

“Nucleic acids” are any of a group of macromolecules, either DNA, RNA, or variants thereof, that carry genetic information that may direct cellular functions. Nucleic acids may have enzyme-like activity (for instance ribozymes) or may be used to inhibit gene expression in a subject (for instance RNAi). Nucleic acids for use herein include single-stranded, double-stranded, linear or circular nucleic acids. Additionally, nucleic acid variants for use herein include aptamers, PNA, LNA, Morpholino, or other non-natural variants of nucleic acids.

In certain embodiments, nucleic acids for use herein include those that encode an amino acid A, C, D, E, F, G, H, I, K, L, M, N, P, Q, R, S, T, V, W, and Y.

“Patient response” or “response” can be assessed using any endpoint indicating a benefit to the patient, including, without limitation, (1) inhibition, to some extent, of disease progression, including stabilization, slowing down and complete arrest; (2) reduction in the number of disease episodes and/or symptoms; (3) inhibition (i.e., reduction, slowing down or complete stopping) of a disease cell infiltration into adjacent peripheral organs and/or tissues; (4) inhibition (i.e. reduction, slowing down or complete stopping) of disease spread; (5) decrease of an autoimmune condition; (6) favorable change in the expression of a biomarker associated with the disorder; (7) relief, to some extent, of one or more symptoms associated with a disorder; (8) increase in the length of disease-free presentation following treatment; or (9) decreased mortality at a given point of time following treatment.

As used herein, the term “peptide” is any peptide comprising two or more amino acids. The term peptide includes short peptides (e.g., peptides comprising between 2-14 amino acids), medium length peptides (15-50) or long chain peptides (e.g., proteins). The terms peptide, polypeptide, medium length peptide and protein may be used interchangeably herein. As used herein, the term “peptide” is interpreted to mean a polymer composed of amino acid residues, related naturally occurring structural variants, and synthetic non-naturally occurring analogs thereof linked via peptide bonds, related naturally-occurring structural variants, and synthetic non-naturally occurring analogs thereof. Synthetic peptides can be synthesized, for example, using an automated peptide synthesizer. Peptides can also be synthesized by other means such as by cells, bacteria, yeast or other living organisms. In certain embodiments, peptides may contain a combination of amino acids from both the 20 gene-encoded amino acids and other modified or synthetic amino acids as shown below in Table 1. In certain embodiments, peptides include amino acids selected from A, C, D, E, F, G, H, I, K, L, M, N, P, Q, R, S, T, V, W, and Y. In other embodiments, peptides include amino acids other than the 20 gene-encoded amino acids. Peptides include those modified either by natural processes, such as processing and other post-translational modifications, but also by chemical modification techniques. Such modifications are well-known in the art. Modifications can occur anywhere in a peptide, including the peptide backbone, the amino acid side chains, the amino or carboxyl termini, glycosylation, phosphorylation, lipidation, acetate attachment, amide attachment, or other hydrocarbon attachments.

TABLE 1

| Full Name | 3 Letter | 1 Letter |
|---|---|---|
| Alanine | Ala | A |
| Arginine | Arg | R |
| Asparagine | Asn | N |
| Aspartate | Asp | D |
| Aspartate or Asparagine | Asx | B |
| Cysteine | Cys | C |
| Glutamate | Glu | E |
| Glutamine | Gln | Q |
| Glutamate or Glutamine | Glx | Z |
| Glycine | Gly | G |
| Histidine | His | H |
| Isoleucine | Ile | I |
| Leucine | Leu | L |
| Lysine | Lys | K |
| Methionine | Met | M |
| Phenylalanine | Phe | F |
| Proline | Pro | P |
| Serine | Ser | S |
| Threonine | Thr | T |
| Tryptophan | Trp | W |
| Tyrosine | Tyr | Y |
| Valine | Val | V |

As used herein, a “pharmaceutically acceptable carrier” or “therapeutic effective carrier” is aqueous or nonaqueous (solid), for example alcoholic or oleaginous, or a mixture thereof, and can contain a surfactant, emollient, lubricant, stabilizer, dye, perfume, preservative, acid or base for adjustment of pH, a solvent, emulsifier, gelling agent, moisturizer, stabilizer, wetting agent, time release agent, humectant, or other component commonly included in a particular form of pharmaceutical composition. Pharmaceutically acceptable carriers include, for example, aqueous solutions such as water or physiologically buffered saline or other solvents or vehicles such as glycols, glycerol, and oils such as olive oil or injectable organic esters. A pharmaceutically acceptable carrier can include physiologically acceptable compounds that act, for example, to stabilize or to increase the absorption of specific modulator(s). This includes, for example, carbohydrates, such as glucose, sucrose or dextrans, antioxidants such as ascorbic acid or glutathione, chelating agents, low molecular weight proteins or other stabilizers or excipients.

The term “pharmaceutical dose” or “pharmaceutical dosage form,” refers to physically discrete units suitable as unitary dosages for humans and other mammals, each unit comprising a predetermined quantity of agents in an amount calculated sufficient to produce the desired effect in association with an acceptable diluent, carrier, or vehicle of a formulation. The specifications for the unit dosage forms may depend on the particular albumin form employed, the effect to be achieved, the route of administration and the pharmacodynamics associated with the mammal.

“PNA” refers to peptide nucleic acids with a chemical structure similar to DNA or RNA. Peptide bonds are used to link the nucleotides or nucleosides together.

“Stringency” of hybridization reactions is readily determinable by one of ordinary skill in the art, and generally is an empirical calculation dependent upon probe length, washing temperature, and salt concentration. In general, longer probes require higher temperatures for proper annealing, while shorter probes need lower temperatures.

“Stringent conditions” or “high stringency conditions”, as defined herein, can be identified by those that: (1) employ low ionic strength and high temperature for washing, for example 0.015 M sodium chloride/0.0015 M sodium citrate/0.1% sodium dodecyl sulfate at 50° C.; (2) employ during hybridization a denaturing agent, such as formamide, for example, 50% (v/v) formamide with 0.1% bovine serum albumin/0.1% Ficoll/0.1% polyvinylpyrrolidone/50 Mm sodium phosphate buffer at pH 6.5 with 750 Mm sodium chloride, 75 Mm sodium citrate at 42° C.; or (3) overnight hybridization in a solution that employs 50% formamide, 5×SSC (0.75 M NaCl, 0.075 M sodium citrate), 50 Mm sodium phosphate (pH 6.8), 0.1% sodium pyrophosphate, 5×Denhardt's solution, sonicated salmon sperm DNA (50 μL/mL), 0.1% SDS, and 10% dextran sulfate at 42° C., with a 10 minute wash at 42° C. in 0.2×SSC (sodium chloride/sodium citrate) followed by a 10 minute high-stringency wash consisting of 0.1×SSC containing EDTA at 55° C.

As used herein, “treatment” refers to clinical intervention in an attempt to alter the course of the health of an individual or cell being treated, and can be performed before or during the course of clinical pathology. Desirable effects of treatment include preventing the occurrence or recurrence of a disease or a condition or symptom thereof, alleviating a condition or symptom of the disease, diminishing any direct or indirect pathological consequences of the disease, decreasing the rate of disease progression, ameliorating or palliating the disease state, or achieving remission or improved prognosis. In some embodiments, methods and compositions are useful in attempts to delay development of a disease or disorder.

It is intended that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

### Polynucleotides Encoding Albumin Variants

The present disclosure provides a nucleic acid encoding an albumin variant polypeptide. In certain embodiments, a nucleic acid encodes an albumin variant that retains at least a portion of the albumin functional activity. The nucleic acid may be DNA molecules, RNA molecules, aptamers (single-stranded or double-stranded), DNA or RNA oligonucleotides, larger DNA molecules that are linear or circular, oligonucleotides that are used for RNA interference (RNAi), variations of DNA such as substitution of DNA/RNA hybrid molecules, synthetic DNA-like molecules such as PNA or other nucleic acid derivative molecules.

In some embodiments, an albumin variant nucleic acid is synthesized using known methods such as enzyme generation. In exemplary embodiments, the enzymes may include DNA polymerases, RNA polymerases, ligases, and DNA repair enzymes. In another embodiment, a nucleic acid is generated by a polymerase chain reaction (PCR) protocol. In other embodiments, the nucleic acids are chemically synthesized using techniques, such as solid-phase nucleic acid synthesizers. Exemplary chemistries include phosphodiester synthesis, phosphotriester synthesis, and others well-known in the art. See, e.g., Reese, Colin B., Organic & Biomolecular Chem. 3(21): 3851 (2005). The skilled artisan would understand that any techniques for synthesizing the nucleic acids and derivatives disclosed herein may be used.

Another aspect includes an albumin variant polypeptide having at least about a 90% sequence identity to SEQ ID NO:1. Other embodiments include a nucleic acid having a sequence that encodes SEQ ID NO:2. In another embodiment, an mRNA encoding SEQ ID NO:2 is delivered to a patient as part of a treatment for neuroinflammatory or neurodegenerative symptoms. Another embodiment includes a nucleic acid that hybridizes with high stringency to a nucleic acid encoding SEQ ID NO:2. In other embodiments, a nucleic acid encoding an albumin variant polypeptide is delivered to an individual via a viral vector, as a naked nucleic acid, or in a transformed cell.

In certain embodiments, nucleic acids encoding an albumin variant polypeptide are administered to a patient in a cell-dependent manner. In certain embodiments, the albumin variants or nucleic acids encoding them are delivered using transfected autologous patient cells. In other embodiments, an albumin variant polypeptide is delivered by intrathecal, intramuscular, intravascular, subcutaneous, intracranial, intraocular injection or inhaled routes. In specific embodiments, a nucleic acid encodes a variant albumin polypeptide having at least about a 90% sequence identity to SEQ ID NO:2. In certain specific embodiments, an albumin variant polypeptide is encoded by a nucleic acid that hybridizes with high stringency to a nucleic acid having SEQ ID NO:2.

Another aspect includes a liquid or powder formulation, comprising a non-viral albumin variant polypeptide. In some embodiments, the albumin variant polypeptide dose range is based on the selection of an albumin form and associated properties. For example, plasmid backbone, promoter strength, and size, etc. In certain embodiments, the copy number ranges from about 500 mM to about 10 nM per dose, depending on the use. Other embodiments comprise a copy number from about 50 mM to about 1 nM per dose. Other embodiments comprise a copy number from about 5 mM to about 100 pM per dose. Other embodiments comprise a copy number from about 100 nM to about 10 pM per dose. Other embodiments comprise a copy number from about 10 nM to about 1 pM per dose.

Another aspect includes a viral particle comprising a nucleic acid that encodes an albumin variant polypeptide. Certain embodiments comprise a liquid or powder formulation comprising the viral particle. The variant albumin polypeptide dose can range based on selection of virus. Generally recommended are dose ranges from about 5×10⁹ PFU/Ml to about 1×10³PFU/Ml per dose, depending on the use. Some compositions may comprise albumins from about 5×10⁹ PFU/Ml to about 1×10⁸ PFU/Ml per dose. Some compositions may comprise albumins from about 0.9×10⁸ PFU/Ml to about 1×10⁶ PFU/Ml per dose. Other compositions may comprise albumins from about 0.9×10⁶ PFU/Ml to about 1×10⁵ PFU/Ml per dose. Yet other compositions may comprise albumins from about 0.9×10⁵ PFU/Ml to about 1×10³ PFU/Ml per dose.

Another aspect includes methods of increasing expression of an albumin variant polypeptide, comprising recombinantly preparing the albumin variant polypeptide, for example, by DNA techniques. Exemplary technologies include homologous recombination, knock-in, ZFNs (zinc finger nucleases), TALENs (transcription activator-like effector nucleases), CRISPR (clustered regularly interspaced short palindromic repeats)/Cas9, and other site-specific nuclease technologies. These techniques enable double-strand DNA breaks at desired locus sites. These controlled double-strand breaks promote homologous recombination at the specific locus sites. This process focuses on targeting specific sequences of nucleic acid molecules, such as chromosomes, with endonucleases that recognize and bind to the sequences and induce a double-stranded break in the nucleic acid molecule. The double-strand break is repaired either by an error-prone non-homologous end-joining (NHEJ) or by homologous recombination (HR).

### Albumin Variant Proteins

The present disclosure provides albumin variant polypeptides for incorporation into treatments of neuroinflammatory or neurodegenerative diseases. The albumin variants may function, for example, by increasing the pharmacokinetics, pharmacodynamics, or bioavailability of said treatment. The compositions may be administered on a daily, weekly, monthly or on an as-needed basis to reduce symptoms of disease or to reduce disease progression. Thus, the present disclosure provides a modified or variant serum albumin polypeptide. In some embodiments, the albumin variant comprises an amino acid substitution in domain 1. In other embodiments, the albumin variant polypeptide has at least a 90% sequence identity to SEQ ID NO:2.

In certain embodiments, the albumin variant polypeptide has a three-dimensional fold that is similar or identical to that of the corresponding unmodified albumin, e.g., as described in Sugio et al., Protein Eng. 12(6):439-46 (1999); Bhattacharya et al., J. Biol. Chem. 275:38731 (2000); and Bhattacharya et al., J. Mol. Biol. 303:721 (2000). The foregoing are incorporated herein by reference in their entirety.

The term “albumin” or “serum albumin” is meant to include mammalian and other species sources. Mammalian includes human serum albumin, bovine serum albumin and other mammalian forms of serum albumin. The sequences of various species serum albumin, particularly mammalian species, are known. Exemplary sequences include albumin sequences from Bos taurus (CAA76847, P02769, CAA41735, 229552, AAF28806, AAF28805, AAF28804, AAA51411); Sus scrofa (P08835, CAA30970, AAA30988); Equus caballus (AAG40944, P35747, CAA52194); Ovis aries (P14639, CAA34903); Salmo salar (CAA36643, CAA43187); Gallus gallus (P19121, CAA43098); Felis catus (P49064, 557632, CAA59279, JC4660); Canis familiaris (P49822, 529749, CAB64867). Engineered variations in amino acid sequences, such as substitutions of amino acids, as described herein, can be introduced into serum albumins including HSA and variants of HSA including naturally occurring mutant forms and engineered forms of HSA, as well as those corresponding positions from other species, such as mammalian serum albumins, including, for example, bovine serum albumin, canine serum albumin, murine serum albumin or others.

Certain embodiments include monomeric albumin variant polypeptides. The molecular weight of wild-type serum albumin from humans is about 66.5 kDa. Similarly, albumin variant polypeptides engineered according to methods herein have molecular weights of about 60-70, 65-67, 66-67, 66, 67, or 66.5 kDa.

In certain embodiments, an albumin variant polypeptide includes a domain 2 that affects FcRn binding. In other embodiments, an albumin variant polypeptide optionally further comprises domain 1 or 3 or comprises all three domains. In certain embodiments, an MSA polypeptide comprises a modified domain 1. In other embodiments, an MSA polypeptide comprises modified domains 1 and 2.

HSA Domain 1, as defined herein, is about 22.9 kDa. In certain embodiments, an albumin variant comprises a modified form of domain 1 having about 20-25, 21-23, 20, 21, 22, 23, 24, 25, 26-30, 31-40, 41-50, 51-60, 61-70, 71-80, 81-90 kDa, or larger molecular weight.

In certain embodiments, an albumin variant comprises a mass of about 66.5 kDa.

As used herein the term “modification” refers to an alteration that physically differentiates the modified molecule from the parent molecule. In one embodiment, an amino acid change in an albumin variant polypeptide prepared according to the methods described herein differentiates it from the corresponding albumin that has not been modified according to the methods described herein, such as wild-type albumin, a naturally occurring mutant albumin or another engineered albumin that does not include the modifications of such albumin variant polypeptide. In another embodiment, an albumin variant polypeptide includes one or more modifications that differentiates the function of the albumin variant polypeptide from the unmodified albumin polypeptide. For example, an amino acid change in an albumin variant polypeptide affects its FcRn binding profile. In other embodiments, an albumin variant polypeptide comprises substitution, deletion, or insertion modifications, or combinations thereof. In another embodiment, an albumin variant polypeptide includes one or more modifications that increases its affinity for FcRn at pH about 5.5 compared to the affinity of the unmodified albumin polypeptide for FcRn at pH about 5.5.

In one embodiment, an albumin variant includes one or more substitutions, insertions, or deletions relative to a corresponding native albumin sequence. In certain embodiments, an albumin polypeptide includes 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31-40, 41 to 50, or 51 or more modifications that affect the FcRn binding profile. In certain embodiments, the albumin variants have enhanced FcRn binding at a pH of about 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5. In other embodiments, the albumin variants have reduced FcRn binding at a pH of about, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, or 8.5.

Another aspect includes recombinant or synthesized albumin variant compositions. In certain embodiments, recombinant albumin variant compositions comprise cell-derived, purified albumin variants. In other embodiments, human albumin variant precursor proteins are purified from an in vitro transfected cell culture.

In certain embodiments, a variant albumin polypeptide comprises post-translational modifications. Exemplary post-translational protein modifications include phosphorylation, acetylation, methylation, ADP-ribosylation, ubiquitination, glycosylation, carbonylation, sumoylation, biotinylation, lipidation, or addition of a polypeptide side chain or of a hydrophobic group. Effects of such non-amino acid elements on the functionality of an albumin may be tested for its biological activity, for example, its ability to bind FcRn.

In certain embodiments, an albumin variant polypeptide may be conjugated to a non-protein agent. Such non-protein agents include, but are not limited to, nucleic acid molecules, chemical agents, organic molecules, etc., each of which may be derived from natural sources, such as for example natural product screening, or may be chemically synthesized.

In certain embodiments, at least one of said amino acid substitutions in an albumin variant is conserved across multiple species. In certain embodiments, a plurality of said amino acid substitutions in an albumin variant are of residues that are conserved across multiple species. In certain embodiments, at least one of said amino acid substitutions in an albumin variant is of a residue that is conserved among serum albumin proteins from human, pig, rat, mouse, dog, rabbit, cow, chicken, donkey, sheep, cat, and horse. In certain embodiments, a plurality of said amino acid substitutions in an albumin variant are of residues that are conserved among serum albumin proteins from human, pig, rat, mouse, dog, rabbit, cow, chicken, donkey, sheep, cat, and horse.

Another aspect includes a protein fusion comprising an albumin variant polypeptide and one or more fusion domains, such as immunoglobulin domains, polyhistidine, Glu-Glu, glutathione S transferase (GST), thioredoxin, protein A, protein G, an immunoglobulin heavy chain constant region (Fc), or maltose binding protein (MBP), which may be used for isolation of the fusion protein by affinity chromatography. For the purpose of affinity purification, relevant matrices for affinity chromatography, such as glutathione-, amylase-, and nickel- or cobalt-conjugated resins are used. Fusion domains also include “epitope tags,” which are usually short peptide sequences for which a specific antibody is available. Useful epitope tags include FLAG, influenza virus haemagglutinin (HA), and c-myc tags. In some cases, the fusion domains have a protease cleavage site, such as for Factor Xa or Thrombin, which allows the relevant protease to partially digest the fusion proteins and thereby liberate the recombinant proteins therefrom. The liberated proteins can then be isolated from the fusion domain by subsequent chromatographic separation.

In some embodiments, modifications at the amino or carboxyl terminus may optionally be introduced into an albumin variant polypeptide. For example, an albumin variant polypeptide can be truncated or acylated on the N-terminus.

In one embodiment, an albumin variant polypeptide comprises a half-life in vivo (for example in human) no less than 10 days, preferably no less than about 14 days, and most preferably no less than 50% of the half-life of the corresponding unmodified albumin polypeptide. In another embodiment, the half-life of an albumin variant is increased by approximately 1.0, 1.5, 2, 2.5, 3, 4, or approximately 5-fold relative to that of the corresponding unmodified albumin polypeptide. In certain embodiments, the half-life of the albumin variant is increased by greater than 5-, or even greater than 10-fold relative to that of the corresponding unmodified albumin polypeptide. In certain embodiments, the half-life of the albumin variant is increased by greater than 20-, 25-, 40-, or greater than 50-fold relative to that of the corresponding unmodified albumin polypeptide.

### Albumin Variant Expression Systems

In certain embodiments, the recombinant nucleic acids encoding an albumin variant polypeptide may be operably linked to one or more regulatory nucleotide sequences in an expression construct. Regulatory nucleotide sequences will generally be appropriate for a host cell used for expression. Numerous types of appropriate expression vectors and suitable regulatory sequences are known in the art for a variety of host cells. Typically, said one or more regulatory nucleotide sequences may include, but are not limited to, promoter sequences, leader or signal sequences, ribosomal binding sites, transcriptional start and termination sequences, translational start and termination sequences, and enhancer or activator sequences. Constitutive or inducible promoters as known in the art are also contemplated. The promoters may be either naturally occurring promoters, or hybrid promoters that combine elements of more than one promoter. An expression construct may be present in a cell on an episome, such as a plasmid, or the expression construct may be inserted in a chromosome. In a specific embodiment, the expression vector includes a selectable marker gene to allow the selection of transformed host cells. Certain embodiments include an expression vector comprising a nucleotide sequence encoding an albumin variant polypeptide operably linked to at least one regulatory sequence. Regulatory sequence for use herein include promoters, enhancers, and other expression control elements. In certain embodiments, an expression vector is designed considering the choice of the host cell to be transformed, the particular albumin variant polypeptide desired to be expressed, the vector's copy number, the ability to control that copy number, or the expression of any other protein encoded by the vector, such as antibiotic markers.

Another aspect includes screening gene products of combinatorial libraries generated by the combinatorial mutagenesis of a nucleic acid described herein. Such screening methods include, for example, cloning the gene library into replicable expression vectors, transforming appropriate cells with the resulting library of vectors, and expressing the combinatorial genes under conditions to form such library. The screening methods optionally further comprise detecting a desired activity and isolating a product detected. Each of the illustrative assays described below are amenable to high-throughput analysis as necessary to screen large numbers of degenerate sequences created by combinatorial mutagenesis techniques.

Certain embodiments include expressing a nucleic acid in microorganisms. One embodiment includes expressing a nucleic acid in a bacterial system, for example, in Bacillus brevis, Bacillus megaterium, Bacillus subtilis, Caulobacter crescentus, Escherichia coli and their derivatives. Exemplary promoters include the 1-arabinose inducible araBAD promoter (PBAD), the lac promoter, the 1-rhamnose inducible rhaP BAD promoter, the T7 RNA polymerase promoter, the trc and tac promoter, the lambda phage promoter Pl, and the anhydrotetracycline-inducible tetA promoter/operator.

Other embodiments include expressing a nucleic acid in a yeast expression system. Exemplary promoters used in yeast vectors include the promoters for 3-phosphoglycerate kinase (Hitzeman et al., J. Biol. Chem. 255:2073 (1980)); other glycolytic enzymes (Hess et al., J. Adv. Enzyme Res. 7:149 (1968); Holland et al., Biochemistry 17:4900 (1978). Others promoters are from, e.g., enolase, glyceraldehyde-3-phosphate dehydrogenase, hexokinase, pyruvate decarboxylase, phosphofructokinase, glucose-6-phosphate isomerase, 3-phosphoglycerate mutase, pyruvate kinase, triosephosphate isomerase, phosphoglucose isomerase, glucokinase alcohol oxidase I (AOX1), alcohol dehydrogenase 2, isocytochrome C, acid phosphatase, degradative enzymes associated with nitrogen metabolism, and the aforementioned glyceraldehyde-3-phosphate dehydrogenase, and enzymes responsible for maltose and galactose utilization. Any plasmid vector containing a yeast-compatible promoter and termination sequences, with or without an origin of replication, is suitable. Certain yeast expression systems are commercially available, for example, from Clontech Laboratories, Inc. (Palo Alto, Calif., e.g. Pyex 4T family of vectors for S. cerevisiae), Invitrogen (Carlsbad, Calif., e.g. Ppicz series Easy Select Pichia Expression Kit) and Stratagene (La Jolla, Calif., e.g. ESP™ Yeast Protein Expression and Purification System for S. pombe and Pesc vectors for S. cerevisiae).

Other embodiments include expressing a nucleic acid in mammalian expression systems. Examples of suitable mammalian promoters include, for example, promoters from the following genes: ubiquitin/S27a promoter of the hamster (WO 97/15664), Simian vacuolating virus 40 (SV40) early promoter, adenovirus major late promoter, mouse metallothionein-I promoter, the long terminal repeat region of Rous Sarcoma Virus (RSV), mouse mammary tumor virus promoter (MMTV), Moloney murine leukemia virus Long Terminal repeat region, and the early promoter of human Cytomegalovirus (CMV). Examples of other heterologous mammalian promoters are the actin, immunoglobulin or heat shock promoter(s). In a specific embodiment, a yeast alcohol oxidase promoter is used.

In additional embodiments, promoters for use in mammalian host cells can be obtained from the genomes of viruses such as polyoma virus, fowlpox virus (UK 2,211,504 published 5 Jul. 1989), bovine papilloma virus, avian sarcoma virus, cytomegalovirus, a retrovirus, hepatitis-B virus and Simian Virus 40 (SV40). In further embodiments, heterologous mammalian promoters are used. Examples include the actin promoter, an immunoglobulin promoter, and heat-shock promoters. The early and late promoters of SV40 are conveniently obtained as an SV40 restriction fragment which also contains the SV40 viral origin of replication. Fiers et al., Nature 273: 113-120 (1978). The immediate early promoter of the human cytomegalovirus is conveniently obtained as a HindIII E restriction fragment. Greenaway, P. J. et al., Gene 18: 355-360 (1982). The foregoing references are incorporated by reference in their entirety.

Other embodiments include expressing a nucleic acid in insect cell expression systems. Eukaryotic expression systems employing insect cell hosts may rely on either plasmid or baculoviral expression systems. Typical insect host cells are derived from the fall army worm (Spodoptera frugiperda). For expression of a foreign protein these cells are infected with a recombinant form of the baculovirus Autographa californica nuclear polyhedrosis virus which has the gene of interest expressed under the control of the viral polyhedron promoter. Other insects infected by this virus include a cell line known commercially as “High 5” (Invitrogen) which is derived from the cabbage looper (Trichoplusia ni). Another baculovirus sometimes used is the Bombyx mori nuclear polyhedorsis virus which infect the silk worm (Bombyx mori). Numerous baculovirus expression systems are commercially available, for example, from Thermo Fisher (Bac-N-Blue™k or BAC-TO-BAC™ Systems), Clontech (BacPAK™ Baculovirus Expression System), Novagen (Bac Vector System™), or others from Pharmingen or Quantum Biotechnologies. Another insect cell host is the common fruit fly, Drosophila melanogaster, for which a transient or stable plasmid based transfection kit is offered commercially by Thermo Fisher (The DES™ System).

In some embodiments, cells are transformed with vectors that express a nucleic acid described herein. Transformation techniques for inserting new genetic material into eukaryotic cells, including animal and plant cells, are well known. Viral vectors may be used for inserting expression cassettes into host cell genomes. Alternatively, the vectors may be transfected into the host cells. Transfection may be accomplished by calcium phosphate precipitation, electroporation, optical transfection, protoplast fusion, impalefection, and hydrodynamic delivery.

Certain embodiments include expressing a nucleic acid encoding an albumin variant polypeptide in mammalian cell lines, for example Chinese hamster ovary cells (CHO) and Vero cells. The method optionally further comprises recovering the albumin variant polypeptide.

### Formulations

Another aspect includes a pharmaceutical formulation comprising an albumin variant disclosed herein. In some embodiments, an albumin variant and a second therapeutic compound are the only active ingredients. In other embodiments, the albumin variants are formulated with a plurality active ingredients. In certain embodiments, the pharmaceutical composition comprises an albumin variant fused to another peptide or protein. In other embodiments, the pharmaceutical composition comprises an albumin variant associated with a non-peptide therapeutic compound. In other embodiments, the non-peptide therapeutic compound is covalently attached to a portion of the albumin variant.

In certain embodiment, a pharmaceutical composition comprises an albumin variant and a pharmaceutically acceptable excipient, carrier, diluent or vehicle. Certain embodiments include powders, liquids, gels, pastes, suspensions, emulsions, or gaseous forms of the pharmaceutical composition. Other embodiments include a dosage form such as a tablet, capsule, caplet, powder, granule, ointment, crème, solution, suspension, emulsion, suppository, injection, inhalant, gel, particle, or aerosol. In other embodiments, the formulations are administered as disclosed herein. In other embodiments, albumin variants are administered in a free form, as pharmaceutically acceptable salts, in a time-release formulation, sequentially in a discrete manner, or in combination with other pharmaceutically active compounds.

In some embodiments, an albumin variant is administered to a patient by intrathecal, intramuscular, intravascular, subcutaneous, intracranial, or intraocular injection. In another embodiment, the albumin variant is provided in liquid and powder formulations at amounts ranging from about 1,000 mg/kg to about 10 mg/kg per dose, depending on the method of administration, potency and use. Some formulations may comprise recombinant albumin variants from about 1,000 mg to about 5 mg per dose.

In other embodiments, the periodicity of dosing varies based on patient needs. In certain embodiments, the dosing schedule is approximately: weekly, bi-weekly, monthly, every 6 weeks or every other month.

Exemplary drug formulations include aqueous solutions, organic solutions, powder formulations, solid formulations and mixed phase formulations.

In certain embodiments, pharmaceutical compositions comprise an albumin variant and a pharmaceutically acceptable carrier, adjuvant or vehicle. Pharmaceutically acceptable carriers, adjuvants and vehicles that may be used in the pharmaceutical compositions include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

Pharmaceutically acceptable salts retain the desired biological activity of the therapeutic composition without toxic side effects. Examples of such salts are (a) acid addition salts formed with inorganic acids, for example, hydrochloric acid, hydrobromic acid, sulfuric acid, phosphoric acid, nitric acid and the like/and salts formed with organic acids such as, for example, acetic acid, trifluoroacetic acid, tartaric acid, succinic acid, maleic acid, fumaric acid, gluconic acid, citric acid, malic acid, ascorbic acid, benzoic acid, tanic acid, pamoic acid, alginic acid, polyglutamic acid, naphthalenesulfonic acid, naphthalene disulfonic acid, polygalacturonic acid and the like; (b) base addition salts or complexes formed with polyvalent metal cations such as zinc, calcium, bismuth, barium, magnesium, aluminum, copper, cobalt, nickel, cadmium, and the like; or with an organic cation formed from N,N′-dibenzylethylenediamine or ethenediamine; or (c) combinations of (a) and (b), e.g. a zinc tannate salt and the like.

In certain embodiments, a pharmaceutical composition is administered by subcutaneous, transdermal, oral, parenteral, inhalation, ocular, topical, rectal, nasal, buccal (including sublingual), vaginal, or implanted reservoir modes. The term parenteral as used herein includes subcutaneous, intracutaneous, intravenous, intramuscular, intraarticular, intrasynovial, intrasternal, intrathecal, intralesional, and intracranial injection or infusion techniques.

In certain embodiments, a pharmaceutical composition comprising an albumin variant as an active ingredient, or pharmaceutically acceptable salt thereof, in a mixture with a pharmaceutically acceptable, non-toxic component is prepared for parenteral administration, particularly in the form of liquid solutions or suspension; for oral or buccal administration, particularly in the form of tablets or capsules; for intranasal administration, particularly in the form of powders, nasal drops, evaporating solutions or aerosols; for inhalation, particularly in the form of liquid solutions or dry powders with excipients, defined broadly; for transdermal administration, particularly in the form of a skin patch or microneedle patch; and for rectal or vaginal administration, particularly in the form of a suppository.

The compositions may conveniently be administered in unit dosage form and may be prepared by any of the methods well-known in the pharmaceutical art, for example, as described in Remington's Pharmaceutical Sciences, 17^{th} ed., Mack Publishing Co., Easton, Pa. (1985), incorporated herein by reference in its entirety. Formulations for parenteral administration may contain as excipients sterile water or saline alkylene glycols such as propylene glycol, polyalkylene glycols such as polyethylene glycol, saccharides, oils of vegetable origin, hydrogenated napthalenes, serum albumin or other nanoparticles (as used in Abraxane™, American Pharmaceutical Partners, Inc. Schaumburg, Ill.), and the like. For oral administration, the formulation can be enhanced by the addition of bile salts or acylcarnitines. Formulations for nasal administration may be solid or solutions in evaporating solvents such as hydrofluorocarbons, and may contain excipients for stabilization, for example, saccharides, surfactants, submicron anhydrous alpha-lactose or dextran, or may be aqueous or oily solutions for use in the form of nasal drops or metered spray. For buccal administration, typical excipients include sugars, calcium stearate, magnesium stearate, pregelatinated starch, and the like.

Delivery of albumin variant therapeutic compounds described herein to a subject over prolonged periods of time, for example, for periods of one week to one year, may be accomplished by a single administration of a controlled release system containing sufficient active ingredient for the desired release period. Various controlled release systems, such as monolithic or reservoir-type microcapsules, depot implants, polymeric hydrogels, osmotic pumps, vesicles, micelles, liposomes, transdermal patches, iontophoretic devices and alternative injectable dosage forms may be utilized for this purpose. Localization at the site to which delivery of the active ingredient is desired is an additional feature of some controlled release devices, which may prove beneficial in the treatment of certain disorders.

In certain embodiments for transdermal administration, delivery across the barrier of the skin would be enhanced using electrodes (e.g., iontophoresis), electroporation, or the application of short, high-voltage electrical pulses to the skin, radiofrequencies, ultrasound (e.g., sonophoresis), microprojections (e.g., microneedles), jet injectors, thermal ablation, magnetophoresis, lasers, velocity, or photomechanical waves. The drug can be included in single-layer drug-in-adhesive, multi-layer drug-in-adhesive, reservoir, matrix, or vapor style patches, or could utilize patchless technology. Delivery across the barrier of the skin could also be enhanced using encapsulation, a skin lipid fluidizer, or a hollow or solid microstructured transdermal system (MTS, such as that manufactured by 3M), jet injectors. Additives to the formulation to aid in the passage of therapeutic compounds through the skin include prodrugs, chemicals, surfactants, cell penetrating peptides, permeation enhancers, encapsulation technologies, enzymes, enzyme inhibitors, gels, nanoparticles and peptide or protein chaperones.

One form of controlled-release formulation contains the albumin variant therapeutic compound or its salt dispersed or encapsulated in a slowly degrading, non-toxic, non-antigenic polymer such as copoly(lactic/glycolic). An albumin variant, or salt thereof, may also be formulated in cholesterol or other lipid matrix pellets, or silastomer matrix implants. Additional slow release, depot implant or injectable formulations will be apparent to the skilled artisan. See, for example, Sustained and Controlled Release Drug Delivery Systems, J R Robinson ed., Marcel Dekker Inc., New York, 1978; and Controlled Release of Biologically Active Agents, R W Baker, John Wiley & Sons, New York, 1987.

An additional form of controlled-release formulation comprises a solution of biodegradable polymer, such as copoly(lactic/glycolic acid) or block copolymers of lactic acid and PEG, in a bioacceptable solvent, which is injected subcutaneously or intramuscularly to achieve a depot formulation. Mixing of an albumin variant described herein with such a polymeric formulation is suitable to achieve very long duration of action formulations.

When formulated for nasal administration, the absorption across the nasal mucous membrane may be further enhanced by surfactants, such as, for example, glycocholic acid, cholic acid, taurocholic acid, ethocholic acid, deoxycholic acid, chenodeoxycholic acid, dehdryocholic acid, glycodeoxycholic acid, cycledextrins and the like in an amount in the range of between about 0.1 and 15 weight percent, between about 0.5 and 4 weight percent, or about 2 weight percent. An additional class of absorption enhancers reported to exhibit greater efficacy with decreased irritation is the class of alkyl maltosides, such as tetradecylmaltoside.

The albumin variant pharmaceutical compositions may be in the form of a sterile injectable preparation, for example, as a sterile injectable aqueous or oleaginous suspension. This suspension may be formulated according to techniques known in the art using suitable dispersing or wetting agents (such as, for example, Tween 80) and suspending agents. The sterile injectable preparation may also be a sterile injectable solution or suspension in a non-toxic parenterally-acceptable diluent or solvent, for example, as a solution in 1,3-butanediol. Among the acceptable vehicles and solvents that may be employed are mannitol, water, Ringer's solution and isotonic sodium chloride solution. In addition, sterile, fixed oils are conventionally employed as a solvent or suspending medium. For this purpose, any bland fixed oil may be employed including synthetic mono- or diglycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant such as Ph. Helv or a similar alcohol.

A pharmaceutical composition comprising an albumin variant may be orally administered in any orally acceptable dosage form including, but not limited to, capsules, tablets, and aqueous suspensions and solutions. In the case of tablets for oral use, carriers that are commonly used include lactose and corn starch. Lubricating agents, such as magnesium stearate, are also typically added. For oral administration in a capsule form, useful diluents include lactose and dried corn starch. When aqueous suspensions are administered orally, the active ingredient is combined with emulsifying and suspending agents. If desired, certain sweetening and/or flavoring and/or coloring agents may be added.

The pharmaceutical compositions may also be administered in the form of suppositories for rectal administration. These compositions can be prepared by mixing a compound provided herein with a suitable non-irritating excipient that is solid at room temperature but liquid at the rectal temperature and therefore will melt in the rectum to release the active components. Such materials include, but are not limited to, cocoa butter, beeswax and polyethylene glycols.

Topical administration of the albumin variant pharmaceutical compositions is especially useful when the desired treatment involves areas or organs readily accessible by topical application. For application topically to the skin, the pharmaceutical composition should be formulated with a suitable ointment containing the active components suspended or dissolved in a carrier. Carriers for topical administration include, but are not limited to, mineral oil, liquid petroleum, white petroleum, propylene glycol, polyoxyethylene polyoxypropylene compound, emulsifying wax and water. Alternatively, the pharmaceutical composition can be formulated with a suitable lotion or cream comprising an albumin variant suspended or dissolved in a carrier. Suitable carriers include, but are not limited to, mineral oil, sorbitan monostearate, polysorbate 60, cetyl esters wax, cetearyl alcohol, 2-octyldodecanol, benzyl alcohol and water. The pharmaceutical compositions of the present disclosure may also be topically applied to the lower intestinal tract by rectal suppository formulation or in a suitable enema formulation. Topical transdermal patches are also included herein.

In certain embodiments, a pharmaceutical composition comprising an albumin variant may be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other solubilizing or dispersing agents known in the art.

Articles of Manufacture and Kits

Another aspect includes a pharmaceutical package or kit comprising one or more containers comprising an albumin variant, for example as a pharmaceutically acceptable formulation. In a specific embodiment, the formulations comprise an albumin variant or fusion that was recombinantly fused, chemically conjugated to, or co-formulated with another moiety. A specific embodiment includes a single dose vial comprising the formulation as a sterile liquid. Formulations may be supplied in vials such as 3 cc USP Type I borosilicate amber vials (West Pharmaceutical Services—Part No. 6800-0675) with a target volume of 1.2 ml. Exemplary containers include, but are not limited to, vials, bottles, pre-filled syringes, Intravenous (IV) bags, blister packs (comprising one or more pills). Optionally associated with such container(s) can be a notice in the form prescribed by a governmental agency regulating the manufacture, use or sale of pharmaceuticals or biological products, which notice reflects approval by the agency of manufacture, use or sale for human diagnosis and/or administration, such as a package insert.

In certain embodiments, kits comprising an albumin variant are also provided that are useful for various purposes, e.g., increasing serum half-life. For isolation and purification of a reagent, the kit may comprise an albumin variant or fusion coupled to beads (e.g., sepharose beads). Kits may be provided that comprise an albumin variant or fusion for detection and quantitation of a target in vitro, e.g. in an Enzyme Linked Immunosorbant Assay (ELISA), a Western blot, or in vivo, for example as a biomarker. As with the article of manufacture, the kit comprises a container and a label or package insert on or associated with the container. The container comprises a pharmaceutically acceptable formulation comprising an albumin variant. Additional containers may be included that comprise, e.g., diluents and buffers, control or diagnostic reagents. The label or package insert may provide a description of the composition as well as instructions for the intended in vitro, in vivo or diagnostic use.

In order that the invention described herein may be more fully understood, the following examples are set forth. It should be understood that these examples are for illustrative purposes only and are not to be construed as limiting this invention in any manner.

### EXAMPLES

### Example 1: Generating Mutant Libraries

The wild-type mature albumin gene (SEQ ID NO:1) was cloned into a yeast display vector, which displays the protein of interest as a C-terminal fusion with the yeast cell wall protein Aga2p under the Gall-10 promoter. See Chao, et al. Nature Protocols 1:755 (2006). NheI and BamHI restriction sites flanked the gene at the 5′ and 3′ ends, respectively, to facilitate cloning. A HindIII restriction site was added shortly after the end of domain I in order to facilitate cloning of this fragment via NheI-HindIII.

To enhance the FcRn-binding properties of albumin, a library of domain 1 mutants was constructed using error-prone PCR using standard methods (for example, see Boder and Wittrup, Methods Enzymol. 328:430-44 (2000), incorporated by reference herein in its entirety). To obtain a range of mutational frequencies, six different mutagenesis PCR reactions were performed on a template plasmid containing the full-length wild-type human serum albumin gene.

In each reaction, the yeast codon-optimized wild-type albumin gene was used as a template DNA. Errors were incorporated by the inclusion of the nucleotide analogs 8-oxo-dGTP and dPTP (TriLink, San Diego, Calif.) to the reactions. The frequency of mutations was tuned by titrating the concentration of analogs and utilizing different numbers of PCR cycles. The six reactions conditions were as follows: reaction 1, 8 cycles with 2 μM analogs; reaction 2, 12 cycles with 2 μM analogs; reaction 3, 20 cycles with 2 μM analogs; reaction 4, 8 cycles with 20 μM analogs; reaction 5, 12 cycles with 20 μM analogs; reaction 6, 20 cycles with 20 μM analogs.

Each reaction contained 1 ng wild-type plasmid, AmpliTAQ Gold™ (Thermo Fisher Scientific, Waltham, Mass.) diluted to 1×, the appropriate concentration of analogs, and 0.5 μM amplification primers targeted to domain 1:

Forward:
CTAGTGGTGGAGGAGGCTCTGGTGGAGGCGGTAGCGGAGGCGGAGGGTCG
GCTAGC
Reverse:
TCGCCACAGCCCAAGCCTTAAAGGCCCTTTCTCCAAACTTTTGTAAGCTT
GCACA

In each cycle, the reaction was heated to 95° C. for 30 seconds followed by annealing at 55° C. for 30 seconds, and finally amplification at 72° C. for 90 seconds. Mutated PCR products were purified on agarose gels to ensure removal of any wild-type parental plasmid DNA. A second round of amplification reactions were performed using the gel-purified, mutated insert as template. The PCR conditions for the amplification reactions were the same as above with the following exceptions: a) no analogs were included; b) template amounts were 2 μl (20 cycle), 5 μl (12 cycle), and 10 μl (8 cycle); and c) 30 cycles were performed in the PCR.

The following amounts of agarose gel-purified, amplified DNA was pooled and concentrated by ethanol precipitation: 7.5 μg (20 cycle/20 μM analogs), 6 μg (20 cycle/0 μM analogs), 6 μg (12 cycle/20 μM analogs), 4.5 μg (12 cycle/2 μM analogs), 3 μg (8 cycle/20 μM analogs), and 3 μg (8 cycle/2 μM analogs). The DNA was resuspended in double deionized water at a final concentration of 1 mg/ml.

### Example 2: Yeast Transformed with the Mutant DNA Library for Surface Display

Once the mutant DNA library was created and amplified, it was then transformed into yeast to display the encoded proteins on the yeast cells surfaces. Surface expression of the library at the protein level allowed the biochemical properties of the library members to be assayed while permitting clones with desired characteristics to be isolated.

The methods for transforming a mutant DNA library into yeast are described, for example, in Chao, et al. Nature Protocols 1:755 (2006) and Colby, et al. Methods in Enzymol. 388: 348 (2004), incorporated by reference herein in its entirety. The library DNA was mixed with linearized yeast display plasmid at an approximate ratio of 3:1 (wt/wt). The DNA was electroporated into freshly made electrocompetent EBY100 yeast where the fragments were assembled by homologous recombination in vivo. After electroporation, yeast was recovered for 1 hour in liquid YPD media while shaking at 30° C. After recovery, yeast was transferred to selective SD-CAA media (Technova Cat. No. 2S0540-02, Holister, Calif.) and grown overnight at 30° C. The library was split once to an OD₆₀₀ of ˜1 to dilute out non-transformants, subsequently grown to saturation, and expression was then induced in SG-CAA media at OD₆₀₀ 1 at either 20° C. or 30° C. for 16-24 hr.

### Example 3: Selection of High Affinity Variants

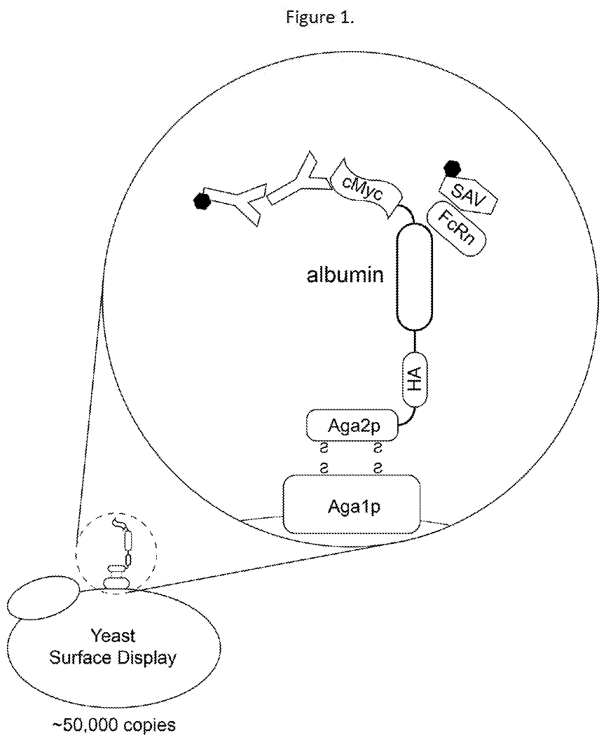

Several methods exist for isolating mutants with desired properties from the yeast displayed libraries. These methods have been described, for example in Chao, et al. Nature Protocols 1:755 (2006), incorporated by reference herein in its entirety. The method used was modified from Chao and is shown in FIG. 1.

In the yeast display construct (FIG. 1), the albumin variants were fused with the yeast cell wall protein Aga2p and thus tethered to the cell surface. A flanking c-Myc epitope tag was included. Yeast expressing the albumin variants were labeled with a chicken polyclonal anti-C-myc antibody (Thermo Fisher Scientific, Waltham, Mass., Cat. No. A-21281) bound to the albumin/C-myc fusions at the cell surface and visualized with Alexa Fluor® 488 conjugated goat anti-chicken polyclonal antibodies (Thermo Fisher Scientific, Cat. No. A-11039). Binding was simultaneously assessed using biotinylated FcRn alpha chain extracellular domain/β₂ microglobulin heterodimers (R&D Systems, Minneapolis, Minn., Cat. No. 8639-FC-050) and fluorescently-labeled streptavidin (R&D Systems Cat. 8639-FC). Varying concentrations of biotinylated FcRn were used depending on the sorting round. The biotinylated antigen was detected with fluorophore-tagged streptavidin following a binding and washing step.

Briefly, during the binding step, yeast expressing albumin variants were resuspended in phosphate buffered saline (PBS) pH 5.5 with 0.1% fish gelatin, a 1:1000 dilution of chicken anti-c-Myc, and a fixed concentration of biotinylated FcRn. The anti-c-Myc antibody was used to monitor expression. Binding was simultaneously assessed using biotinylated extracellular FcRn domain and fluorescently-labeled streptavidin (SAV). All subsequent binding and wash steps were performed in the same PBS pH 5.5 plus fish gelatin buffer. The reaction was allowed to proceed at room temperature for at least three hours to allow equilibrium to be reached. After binding, yeast was pelleted, washed once and resuspended in a secondary labeling solution containing a 1:1000 dilution of goat anti-chicken AlexaFluor488 (Thermo Fisher Scientific, Waltham, Mass.) and streptavidin AlexaFluor647 (Thermo Fisher Scientific, Waltham, Mass.). Secondary labeling was performed for 20 minutes on ice, after which cells were pelleted, washed once and stored as pellets on ice prior to sorting.

Yeast displaying albumin variants with high affinity to FcRn were selected using fluorescence activated cell sorting (FACS). Briefly, singlet yeast with the greatest amount of binding for a given level of expression as determined by mean fluorescence intensity corresponding to streptavidin AlexaFluor647 and goat anti-chicken AlexaFluor488 respectively, were sorted. Collected yeast were amplified in liquid SD-CAA culture, induced in SG-CAA (Technova Cat. No. 2S0542-02, Hollister, Calif.), followed by labeling and sorting as described for subsequent rounds of selection.

After each sort round, binding to FcRn at pH 5.5 and at pH 7.4 was monitored. Despite sorting being exclusively done at pH 5.5, little to no binding to FcRn was observed at pH 7.4 throughout the sorts.

After 4 rounds of sorting, the library was shuffled and further mutagenized to select variants with enhanced binding affinities as follows: Plasmid DNA from the enriched pool of albumin domain 1 variants was isolated by Zymoprep™ (Zymo Research, Irvine, Calif.). The domain 1 fragment was amplified by PCR and a wild-type domain 1 was similarly amplified by PCR. Each reaction contained 5 uL of either zymoprep DNA or 1 uL of wild-type plasmid, AmpliTAQ Gold™ (Thermo Fisher Scientific, Waltham, Mass.) diluted to 1×, and 0.5 μM amplification primers (as described above) specific to domain 1. Reactions were cycled 30 times wherein each cycle, the reaction was heated to 95° C. for 30 seconds followed by annealing at 55° C. for 30 seconds, and finally amplification at 72° C. for 90 seconds. Amplified mutated and wild-type DNA were gel-purified and subjected to digestion by DNaseI. Briefly, 6 μg of mutagenized and 1.5 μg of wild-type DNA were combined with 1 unit of DNaseI in 50 mM Tris pH 8 and 10 mM MgCl₂. Digestion was carried out for 3 minutes at 15° C., after which the reaction was heated to 90° C. for 10 to denature the enzymes. Fragments were reassembled by PCR as described with the only difference being the exclusion of any amplification primers. Products from the assembly reaction were used as templates for reactions that included amplification primers. In some cases, 1 μM of nucleotide analogs were added to the amplification reactions to incorporate additional, random mutations on top of the recombined mutations. Amplified DNA was pooled, gel-purified, and concentrated by ethanol precipitation. DNA was resuspended in double deionized water at a final concentration of 1 mg/ml. Yeast were transformed as described above to make a second affinity maturation library.

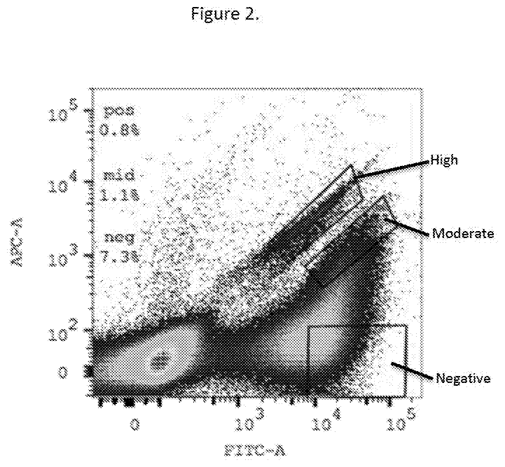

The shuffled library was subjected to 5 rounds of labeling and selection by FACS as described for the original library. Throughout sorting, binding to FcRn at pH 7.4 was monitored. Following the 5^{th} round of sorting, a sub-population of the library was found to bind to FcRn at pH 7.4. For the subsequent 6^{th} round of sorting, yeast was labeled with 1 μM FcRn at pH 7.4 and three distinct sub-populations were observed. From this sort, three sub-populations were collected, as shown in FIG. 2: high binding at pH 7.4 (hereafter to be referred to as pH7.4high pool), moderate binding at pH 7.4 (hereafter to be referred to as pH7.4mid pool), and negative binding at pH 7.4 (hereafter to be referred to as pH7.4neg pool). All three sub-populations retained high binding to FcRn at pH 5.5.

The yeast pool with high binding at pH 5.5 and no binding at pH 7.4, pH7.4neg pool, was subjected to one additional sort round at pH 5.5, and a subset of the subsequent pooled clones were sequenced (hereafter to be referred to as S7 prds).

### Example 4: Improved FcRn-Binding and pH Sensitivity of Enriched Library Pool

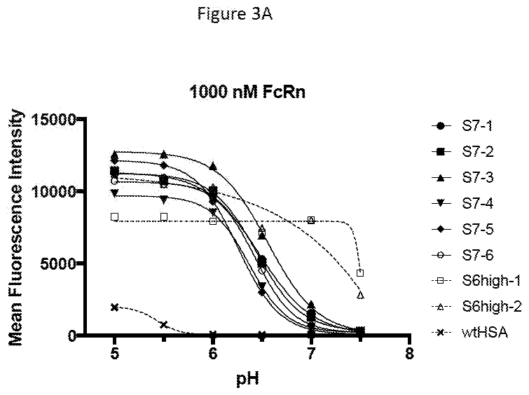

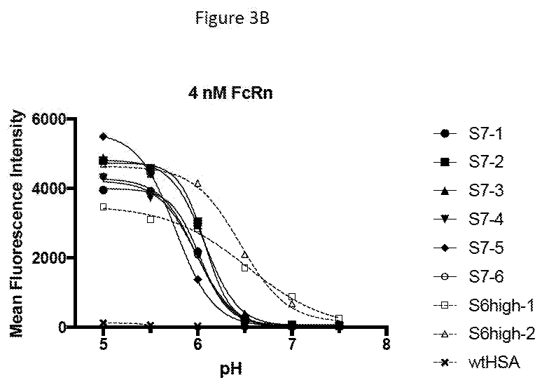

Exemplary yeast pools were tested for binding to FcRn at a pH range of 5.0 to pH 7.4 as described above and compared to wild-type albumin. FIGS. 3A and 3B show variants from the enriched library with substantially enhanced binding at pH 5.0 relative to wild-type albumin, while concurrently displaying negligible affinity to FcRn at pH 7.4. FIG. 3A shows the results when 1000 nM FcRn was used and FIG. 3B shows when 4 nM FcRn was used.

### Example 5: Sequences of Individual Clones

Individual clones comprising the most desirable variants from the enriched libraries were sequenced. Briefly, plasmid DNA was recovered from the enriched pool of yeast by zymoprep (Zymo Research, Irvine, Calif.) and transformed into Top10 E. coli (Invitrogen, Thermo Fisher Scientific, Waltham, Mass.). Individual colonies were selected for sequencing. Exemplary sequences of clones from the pH7.4high, pH7.4mid, and pH7.4neg pools are shown in Tables 2, 3, and 4, respectively. Although there was no sequence convergence for the clones, certain positions along the albumin peptide backbone were identified that had higher numbers of variations from the wild-type sequence.

In the sort 6 pH7.4high pool, the mutation F19L was identified more than 50% of the clones sequenced. Other mutations appearing in at least one clone include Y30C, L31S, Q32R, K41E, A50T, K51R, T76A, D89G, N130D, K162E, E188G, K190E, and S192P. Exemplary identified clones are as follows.

TABLE 2

| Position | 19 | 30 | 31 | 32 | 41 | 50 | 51 | 76 | 89 | 130 | 162 | 188 | 190 | 192 |
|---|---|---|---|---|---|---|---|---|---|---|---|---|---|---|
| wt | F | Y | L | Q | K | A | K | T | D | N | K | E | K | S |
| S6 pos-1 | L | | | | | T | | | | | | G | | |
| S6 pos-4 | | C | S | | | | | | G | | | | | |
| S6 | L | | | R | E | | R | A | | D | E | | E | P |
| pos-10 | | | | | | | | | | | | | | |

In the sort 6 pH7.4mid pool, the mutations L24S, K93E, K190E, L198P, and K199R occur in more than 50% of the unique clones sequenced. The mutations R10G, F19L, I25V, L27F, C34R, and E188G occurred in more than 30% of the unique clones sequenced. Other mutations appearing in at least one clone include S5P, D13G, N18D, K20E, K41E, K41R, V43A, D56G, H67Y, T68A, M87I, E95G, N99D, K106R, N111S, L1112S, I142T, F149S, P152S, P152L, R160G, F165L, K190R, and K199E. Exemplary identified clones are as follows:

TABLE 3

| Position | wt | 5 | 10 | 13 | 18 | 19 | 20 | 24 | 25 | 27 | 34 | 41 | 43 |
|---|---|---|---|---|---|---|---|---|---|---|---|---|---|
| wt | | S | R | D | N | F | K | L | I | F | C | K | V |
| S6 mid-1 | | P | | | | | | S | V | L | R | | |
| S6 mid-2 | | | | | | | | S | | | | | |
| S6 mid-3 | | P | | | D | | | S | V | L | R | | |
| S6 mid-4 | | | | G | D | L | | | | L | R | E | |
| S6 mid-5 | | | G | | | | | S | V | | | R | A |
| S6 mid-8 | | | G | | | | | S | | | | | |
| S6 mid-9 | | | G | | | | | S | | | | | |
| S6 mid-10 | | | | | | L | | | | L | R | | |
| S6 mid-11 | | | G | | | | | S | | | | | |
| S6 mid-18 | | | | | | | | S | | | | | |
| S6 mid-19 | | | | | | L | | S | | | | | |
| S6 mid-20 | | | | G | | L | E | | V | L | R | | |
| Position | wt | 56 | 67 | 68 | 87 | 93 | 95 | 99 | 106 | 111 | 112 | 142 | |
| wt | | D | H | T | M | K | E | N | K | N | L | I | |
| S6 mid-1 | | G | | A | | | | | | | | | |
| S6 mid-2 | | | | | | E | | | | | | | |
| S6 mid-3 | | | | | | E | | | | | | | |
| S6 mid-4 | | G | | | | | | | R | | S | | |
| S6 mid-5 | | | | | | E | | | | | | | |
| S6 mid-8 | | | | | | E | | | | S | | | |
| S6 mid-9 | | | | | I | E | | | | | | | |
| S6 mid-10 | | | | | | | | | | | | | |
| S6 mid-11 | | | | | | E | | | | | | | |
| S6 mid-18 | | | | | | E | | | | | | T | |
| S6 mid-19 | | | Y | | | E | G | D | | | | | |
| S6 mid-20 | | | | | | | | | | | | | |
| Position | wt | 149 | 152 | 160 | 165 | 188 | 190 | 192 | 194 | 198 | 199 | | |
| wt | | F | P | R | F | E | K | S | A | L | K | | |
| S6 mid-1 | | | | | | G | E | | | P | R | | |
| S6 mid-2 | | | S | | | | E | | | P | E | | |
| S6 mid-3 | | | | | | G | E | | | P | R | | |
| S6 mid-4 | | S | | | | | E | P | | | | | |
| S6 mid-5 | | | | | | | | | | P | R | | |
| S6 mid-8 | | | | | | G | E | | | P | R | | |
| S6 mid-9 | | | | G | | G | E | | | P | R | | |
| S6 mid-10 | | | | | | | R | | | P | R | | |
| S6 mid-11 | | | | | | | | P | | P | R | | |
| S6 mid-18 | | | L | | L | | | | T | P | E | | |
| S6 mid-19 | | | | | | | R | | | P | R | | |
| S6 mid-20 | | | | | | | | | | P | R | | |

In the sort 6 pH7.4neg pool, the mutations K93E, K190E, and L198P occurred in more than 50% of the unique clones sequenced. The mutations R10G, F19L, L24S, S192P, and K199R occurred in more than 30% of the unique clones sequenced. Other mutations appearing in at least one clone include D1G, S5P, E6G, V7A, F11S, K12R, D13G, L14P, G15D, E16G, N18D, K20E, K20R, I25V, F27C, F27L, Y30H, Q32R, E37K, V40A, K41E, V43I, N44E, N44E, A50V, K51R, D56G, D56T, E57G, N61S, K64E, T76A, A78T, A88V, A92T, Q94R, E95G, N99S, K106R, N109D, N111D, N111E, E119G, N130D, K136E, Y138H, I142V, I142T, R145G, P152S, F156S, K162R, K162E, A163T, A171V, E184G, E188G, G189R, A191V, S192P, A194T, Q196R, and K199E. Exemplary identified clones are as follows:

TABLE 4

| Position | 1 | 5 | 6 | 7 | 10 | 11 | 12 | 13 | 14 | 15 | 16 | 18 | 19 | 20 | 24 | 25 | |
|---|---|---|---|---|---|---|---|---|---|---|---|---|---|---|---|---|---|
| wt | D | S | E | V | R | F | K | D | L | G | E | N | F | K | L | I | |
| S6 neg-2 | | | | | | | | | | | | | L | E | | | |
| S6 neg-3 | | | | | | | | | | | | | L | | | | |
| S6 neg-4 | | | | | | | | | | | | | L | | | | |
| S6 neg-6 | | | | | G | | | | | | | | L | | S | | |
| S6 neg-7 | | | | | | | | | | | | | L | E | | | |
| S6 neg-8 | | | | A | | | | G | | | | | | E | | | |
| S6 neg-9 | | | | | G | | | | | | | | | | | | |
| S6 neg-10 | | P | | | | | | | | | | D | | | S | V | |
| S6 neg-11 | | | | | G | | | | | | | | | | S | | |
| S6 neg-14 | | | | | | | | | | | | | | | S | V | |
| S6 neg-15 | | | | | G | | | | | | | | | R | | | |
| S6 neg-16 | | | | | | | | G | | | | | L | | S | | |
| S6 neg-18 | | | | | G | | | | | | | | | | S | V | |
| S6 neg-19 | | | | | G | | | | | | | | | | | | |
| S6 neg-20 | | | | | | | | | | | | | L | E | | | |
| S6 neg-21 | | P | | | | | | | | | | | L | | S | | |
| S6 neg-22 | | | | | G | | R | G | | | | | L | | | | |
| S6 neg-23 | | | | | | | | | | | | | | | | | |
| S6 neg-25 | | | | | G | | | | | | | | | | | | |
| S6 neg-26 | | | | | | | | | | | | | L | E | | | |
| S6 neg-28 | | P | | | | | | | | | | | | | | | |
| S6 neg-30 | | | | | | | | | | | | | L | E | | | |
| S6 neg-32 | | | | | G | | | | | | | | | | S | V | |
| S6 neg-33 | | P | | | | | | | P | | | | L | E | | | |
| S6 neg-34 | G | | | | | | | | | | | | | | S | | |
| S6 neg-35 | | | | | | | | | | | | | | | S | | |
| S6 neg-36 | | | | | | | | | | | G | | L | | | | |
| S6 neg-37 | | | | | G | | | | | | | | | | S | | |
| S6 neg-38 | | | | | | | | | | | | | | | | | |
| S6 neg-40 | | | | | | | | | | | | | | | S | | |
| S6 neg-42 | | | | | | | | G | | | | | | E | | | |
| S6 neg-44 | | | G | | | S | | | | | | | | | | | |
| S6 neg-46 | | | | | G | | | | | D | | | L | | | | |
| S6 neg-48 | | P | | | | | | | | | | | L | | | | |
| S6 neg-49 | | | | | G | | | | | | | | | | S | | |
| S6 neg-50 | | | | | G | | | | | | | | | | S | V | |
| Position | 27 | 30 | 32 | 37 | 40 | 41 | 43 | 44 | 50 | 51 | 56 | 57 | 61 | 64 | 76 | 78 | 88 |
| wt | F | Y | Q | E | V | K | V | N | A | K | D | E | N | K | T | A | A |
| S6 neg-2 | | | | | | | | | | | | | | | A | T | |
| S6 neg-3 | | | | | | | | | V | | | | | | | | |
| S6 neg-4 | | | | | A | | | | | | | G | | | A | | |
| S6 neg-6 | | | | | | | | | | | | | | | | | |
| S6 neg-7 | C | | | | | | | | | | G | | | | | | |
| S6 neg-8 | | H | | | | | | | | | | | | | | | |
| S6 neg-9 | | | | | | | | | | R | | | | | | | |
| S6 neg-10 | L | | | | | | | D | | | | | | | A | | |
| S6 neg-11 | | | | | | | | | | | | | | E | | | |
| S6 neg-14 | L | | | | A | | | | | | | | | | | | V |
| S6 neg-15 | | | | | | | | | | | | | | | | | |
| S6 neg-16 | | | | | | | | | | | | | | | | | |
| S6 neg-18 | | | | | | | | | | | | | | | | | |
| S6 neg-19 | | | | | | | | | | | | | | | | | |
| S6 neg-20 | | | | | | | | | | | | | | | | | |
| S6 neg-21 | | | | | | | | | V | | | | | | A | | |
| S6 neg-22 | | | | | | | | | | | | | | | | | |
| S6 neg-23 | | | | | | | | | | | | | | E | | | V |
| S6 neg-25 | | | | | | E | I | | | | | | | | | | |
| S6 neg-26 | L | | | | | | | | | | | | | | | | |
| S6 neg-28 | | | | | | | | | | | | | | | | | V |
| S6 neg-30 | | | | | | | | | | | | | | | | | |
| S6 neg-32 | | | | | | | | | | | | | | | | | |
| S6 neg-33 | L | | | | | E | | | | | | | | | A | | |
| S6 neg-34 | S | | | | | | | | | | | | | | | | V |
| S6 neg-35 | | | | | | | | | | | | | | | | | |
| S6 neg-36 | L | | R | | | E | | | | | | | | | | | |
| S6 neg-37 | | | | | | | | | | | | G | | | | | |
| S6 neg-38 | L | | R | | | | I | | | | | | S | | | T | |
| S6 neg-40 | | | | | | | | | | | | | | | | T | V |
| S6 neg-42 | L | | | | A | | | | | | T | | | | | | |
| S6 neg-44 | L | | | | | E | I | | | | | | | | | | |
| S6 neg-46 | | | | | | | | E | | | | | | | | | |
| S6 neg-48 | C | | | | | | | | | | | | | | A | | |
| S6 neg-49 | | H | | K | | | | | | | | | | | | | |
| S6 neg-50 | | | | | | | | | | | | | | | | | |
| Position | 92 | 93 | 94 | 95 | 99 | 106 | 109 | 111 | 119 | 130 | 136 | 138 | 142 | 145 | | | |
| wt | A | K | Q | E | N | K | N | N | E | N | K | Y | I | R | | | |
| S6 neg-2 | | E | | | | R | | | | | | | | | | | |
| S6 neg-3 | | E | | | | | | | | | | | V | | | | |
| S6 neg-4 | | E | | | | | | | | | | | | | | | |
| S6 neg-6 | | E | R | | | | | | | | | | | | | | |
| S6 neg-7 | | | | | | | | | | | | | | | | | |
| S6 neg-8 | | | | | | R | D | | | | | | T | | | | |
| S6 neg-9 | | E | | | | R | D | | | | | | | | | | |
| S6 neg-10 | | E | | | | | | D | | | | | | | | | |
| S6 neg-11 | | E | | | S | | | | | | | | | | | | |
| S6 neg-14 | | E | | | | | | | | | | | | | | | |
| S6 neg-15 | | E | | | | | | E | | | | | | | | | |
| S6 neg-16 | | E | | | | | D | D | | | | | | | | | |
| S6 neg-18 | | E | | | | | | | | | | | | | | | |
| S6 neg-19 | | E | | | | | | | G | | | | | | | | |
| S6 neg-20 | | E | | | | | | | | | | H | | G | | | |
| S6 neg-21 | | E | | G | | | | | | | E | | | | | | |
| S6 neg-22 | | E | | | | | | | G | | | | | | | | |
| S6 neg-23 | | E | | | | | | | | | | | | | | | |
| S6 neg-25 | T | E | | | | | | | | | | | | | | | |
| S6 neg-26 | | | | | | | | | | | | | | | | | |
| S6 neg-28 | | E | | | | | | | | | | | | | | | |
| S6 neg-30 | | E | | | | | | | | | | | | | | | |
| S6 neg-32 | | E | | | | | | | | D | | | | | | | |
| S6 neg-33 | | | | | | | | | | | | | | | | | |
| S6 neg-34 | | E | | | | | | | G | | | | | | | | |
| S6 neg-35 | | E | | | | | | | | | | | | | | | |
| S6 neg-36 | | | | | | | | | | | | | | | | | |
| S6 neg-37 | | | | | | | | | | | | | | | | | |
| S6 neg-38 | | | | | | | | | | | | | | | | | |
| S6 neg-40 | | E | | | | | | | | | | | | | | | |
| S6 neg-42 | | | | | | | | | | | | | | | | | |
| S6 neg-44 | | | | | | | | | | | | | | | | | |
| S6 neg-46 | | E | | | | | | D | | | | | | | | | |
| S6 neg-48 | | | | | | | | | | | | | | | | | |
| S6 neg-49 | | E | | | | | | | | | | | | | | | |
| S6 neg-50 | | E | | | | | | | G | | | | | | | | |
| Position | 149 | 152 | 156 | 162 | 163 | 171 | 184 | 188 | 189 | 190 | 191 | 192 | 193 | | | | |
| wt | F | P | F | K | A | A | E | E | G | K | A | S | S | | | | |
| S6 neg-2 | S | | | | | | | | | E | | P | | | | | |
| S6 neg-3 | | | | | | | | G | | E | | | | | | | |
| S6 neg-4 | | | | | | | | | R | E | | | | | | | |
| S6 neg-6 | S | | | | | | | | | E | | P | | | | | |
| S6 neg-7 | | | | | | | | | | | | | P | | | | |
| S6 neg-8 | | | | | | | | | | E | | | P | | | | |
| S6 neg-9 | | | | | | | | | | R | | | | | | | |
| S6 neg-10 | S | | | | | | | | | E | | P | | | | | |
| S6 neg-11 | S | | | R | | | | | | E | | P | | | | | |
| S6 neg-14 | S | | S | | | | | | | E | | P | | | | | |
| S6 neg-15 | | | | | | | | G | | E | | | | | | | |
| S6 neg-16 | | | | | | V | | | | | | | | | | | |
| S6 neg-18 | | | | | | | | G | | E | | | | | | | |
| S6 neg-19 | S | | S | | | | | | | E | | P | | | | | |
| S6 neg-20 | | | | | | | | | | E | | | P | | | | |
| S6 neg-21 | S | | | E | | | | | | E | | P | | | | | |
| S6 neg-22 | | | | R | | | | | | | | | | | | | |
| S6 neg-23 | S | | | | | | | | | E | | P | | | | | |
| S6 neg-25 | | | | | | | | | | E | | | | | | | |
| S6 neg-26 | | | | | | | | | | | | | | | | | |
| S6 neg-28 | S | | | | | | | | | E | | | | | | | |
| S6 neg-30 | S | | | | | | | | | E | | P | | | | | |
| S6 neg-32 | | | | | | | | G | | E | | | | | | | |
| S6 neg-33 | | | | | | | | | | E | V | | | | | | |
| S6 neg-34 | S | | | | | | | | | E | | P | | | | | |
| S6 neg-35 | | S | | | | | | | | E | | | | | | | |
| S6 neg-36 | | | | | | | | | | | | | | | | | |
| S6 neg-37 | | | | | | | | G | | E | | | | | | | |
| S6 neg-38 | | | | | | | | | | | | | P | | | | |
| S6 neg-40 | S | | | | | | | | | E | | P | | | | | |
| S6 neg-42 | | | | | | | | | | | | | | | | | |
| S6 neg-44 | | | | | | | | | | E | | | | | | | |
| S6 neg-46 | S | | | | T | | | | | E | | P | | | | | |
| S6 neg-48 | | | | | | | | | | | | | | | | | |
| S6 neg-49 | S | | | | | | | | | E | | P | | | | | |
| S6 neg-50 | | | | | | | G | | | | | | | | | | |
| Position | 194 | 196 | 198 | 199 | | | | | | | | | | | | | |
| wt | A | Q | L | K | | | | | | | | | | | | | |
| S6 neg-2 | | | | | | | | | | | | | | | | | |
| S6 neg-3 | | | P | R | | | | | | | | | | | | | |
| S6 neg-4 | | | | | | | | | | | | | | | | | |
| S6 neg-6 | | | | | | | | | | | | | | | | | |
| S6 neg-7 | | | P | R | | | | | | | | | | | | | |
| S6 neg-8 | | | | | | | | | | | | | | | | | |
| S6 neg-9 | | | P | R | | | | | | | | | | | | | |
| S6 neg-10 | | | | | | | | | | | | | | | | | |
| S6 neg-11 | | | | | | | | | | | | | | | | | |
| S6 neg-14 | | | | | | | | | | | | | | | | | |
| S6 neg-15 | | | P | R | | | | | | | | | | | | | |
| S6 neg-16 | T | | P | E | | | | | | | | | | | | | |
| S6 neg-18 | | | P | R | | | | | | | | | | | | | |
| S6 neg-19 | | | | | | | | | | | | | | | | | |
| S6 neg-20 | | | P | | | | | | | | | | | | | | |
| S6 neg-21 | | | | | | | | | | | | | | | | | |
| S6 neg-22 | | | P | R | | | | | | | | | | | | | |
| S6 neg-23 | | R | | E | | | | | | | | | | | | | |
| S6 neg-25 | | | P | R | | | | | | | | | | | | | |
| S6 neg-26 | | | P | R | | | | | | | | | | | | | |
| S6 neg-28 | | R | | E | | | | | | | | | | | | | |
| S6 neg-30 | | | | | | | | | | | | | | | | | |
| S6 neg-32 | | | P | R | | | | | | | | | | | | | |
| S6 neg-33 | | | P | R | | | | | | | | | | | | | |
| S6 neg-34 | | | | | | | | | | | | | | | | | |
| S6 neg-35 | | | P | E | | | | | | | | | | | | | |
| S6 neg-36 | | | P | R | | | | | | | | | | | | | |
| S6 neg-37 | | | P | R | | | | | | | | | | | | | |
| S6 neg-38 | | | P | R | | | | | | | | | | | | | |
| S6 neg-40 | | | | | | | | | | | | | | | | | |
| S6 neg-42 | | | P | R | | | | | | | | | | | | | |
| S6 neg-44 | | | P | E | | | | | | | | | | | | | |
| S6 neg-46 | | | | | | | | | | | | | | | | | |
| S6 neg-48 | | | P | R | | | | | | | | | | | | | |
| S6 neg-49 | | | | | | | | | | | | | | | | | |
| S6 neg-50 | | | P | R | | | | | | | | | | | | | |

The sort 6 pH7.4neg pool was subjected to one additional sorting round for the highest affinity binders. In the S7 pool, the mutations K93E, F149S, K190E, and S192P occurred in more than 50% of the unique clones sequenced. The mutations F19L, L24S, A88V, Q196R, and K199E occurred in more than 30% of the unique clones sequenced. Other mutations appearing in at least one clone include D1G, H3Y, S5P, R10G, K12R, D13G, N18D, K20E, I25V, F27L, F27S, C34R, A50V, K51R, E60G, K64E, T76A, V77I, A78T, C90W, E95G, C101W, K106R, N111D, V116A, E119G, V120A, E119G, V120A, N130D, K136E, K137R, I142V, I142T, E153G, F156S, F157L, K159R, R160G, K162R, K162E, F165L, Q170R, K174R, D183G, E184G, E188G, L198P, and K199R. Exemplary identified sequences are as follows:

TABLE 5

| Position | | 1 | 3 | 5 | 10 | 12 | 13 | 18 | 19 | 20 | 24 | 25 |
|---|---|---|---|---|---|---|---|---|---|---|---|---|
| wt | wt | D | H | S | R | K | D | N | F | K | L | I |
| HSA D1 shuffle S7-1 | | | | P | | | | | | | | |
| HSA D1 shuffle S7-2 | | | | | | | | | L | E | | |
| HSA D1 shuffle S7-4 | | | | | | | | D | L | | | |
| HSA D1 shuffle S7-5 | | | | | | | | | L | E | | |
| HSA D1 shuffle S7-6 | | | | P | | | | | L | | S | |
| HSA D1 shuffle S7-8 | | | | | | | | | | | | |
| HSA D1 shuffle S7-9 | | | | | G | | | | | | S | |
| HSA D1 shuffle S7-13 | | | | | | | | | | | S | |
| HSA D1 shuffle S7-14 | | | | | G | | | | | | S | |
| HSA D1 shuffle S7-17 | | | | | | | G | | L | | | |
| HSA D1 shuffle S7-20 | | | | | G | | | | | | | |
| HSA D1 shuffle S7-22 | | | | P | | | | | | | | |
| HSA D1 shuffle S7-23 | | G | | | | | | | | | S | |
| HSA D1 shuffle S7-26 | | | | | | | | | | | S | V |
| HSA D1 shuffle S7-27 | | | | | G | | | | | | | |
| HSA D1 shuffle S7-28 | | | | | | | G | | L | | | |
| HSA D1 shuffle S7-29 | | | | | G | R | | | | | S | |
| HSA D1 shuffle S7-30 | | | Y | | | | | | L | E | S | |
| HSA D1 shuffle S7-31 | | | Y | | | | G | | | | S | |
| HSA D1 shuffle S7-32 | | | | | | | | | | | | |
| HSA D1 shuffle S7-33 | | | | | | | | | | | | |
| Position | | 27 | 34 | 50 | 51 | 60 | 64 | 76 | 77 | 78 | 88 | 90 |
| wt | | F | C | A | K | E | K | T | V | A | A | C |
| HSA D1 shuffle S7-1 | | | | | | | | | | | | |
| HSA D1 shuffle S7-2 | | | | | | | | A | | T | | |
| HSA D1 shuffle S7-4 | | L | R | | | | | | | | | |
| HSA D1 shuffle S7-5 | | | | | | | | A | | T | | W |
| HSA D1 shuffle S7-6 | | | | V | | | | A | | | | |
| HSA D1 shuffle S7-8 | | | | | | | E | | | | V | |
| HSA D1 shuffle S7-9 | | | | | | | | | | | | |
| HSA D1 shuffle S7-13 | | | | | | | | | | | V | |
| HSA D1 shuffle S7-14 | | | | | | G | | | | | | |
| HSA D1 shuffle S7-17 | | | | V | | | | | | | | |
| HSA D1 shuffle S7-20 | | | | | | | | | | | V | |
| HSA D1 shuffle S7-22 | | | | | | | | | | | V | |
| HSA D1 shuffle S7-23 | | S | | | | | | | | | V | |
| HSA D1 shuffle S7-26 | | L | | | | | | | | | | |
| HSA D1 shuffle S7-27 | | | | | | | | | | | V | |
| HSA D1 shuffle S7-28 | | | | | R | | | | | | | |
| HSA D1 shuffle S7-29 | | | | | | | | | | | | |
| HSA D1 shuffle S7-30 | | | | | | | | | | | | |
| HSA D1 shuffle S7-31 | | | | V | | | | | | | | |
| HSA D1 shuffle S7-32 | | | | | | | | | | | V | |
| HSA D1 shuffle S7-33 | | | | | | | | | I | | | |
| Position | | 93 | 95 | 101 | 106 | 111 | 116 | 119 | 120 | 130 | 136 | |
| wt | | K | E | C | K | N | V | E | V | N | K | |
| HSA D1 shuffle S7-1 | | E | G | | | D | | | | | | |
| HSA D1 shuffle S7-2 | | E | | | R | | | | | | | |
| HSA D1 shuffle S7-4 | | E | | | | D | | | A | | | |
| HSA D1 shuffle S7-5 | | E | | W | R | | | | | | | |
| HSA D1 shuffle S7-6 | | E | G | | | | | | | | E | |
| HSA D1 shuffle S7-8 | | E | | | | | | | | | | |
| HSA D1 shuffle S7-9 | | E | | | | | | | | D | | |
| HSA D1 shuffle S7-13 | | | | | | D | | | | | | |
| HSA D1 shuffle S7-14 | | E | | | | | | | | | | |
| HSA D1 shuffle S7-17 | | E | | | | | A | | | | | |
| HSA D1 shuffle S7-20 | | E | | | R | D | | G | | | | |
| HSA D1 shuffle S7-22 | | E | | | | | | | | | | |
| HSA D1 shuffle S7-23 | | E | | | | | | G | | | | |
| HSA D1 shuffle S7-26 | | E | | | | | | | | | | |
| HSA D1 shuffle S7-27 | | E | | | | | | | | D | | |
| HSA D1 shuffle S7-28 | | E | | | | | | | | | | |
| HSA D1 shuffle S7-29 | | E | | | | | | | | | | |
| HSA D1 shuffle S7-30 | | | | | | D | | | | | | |
| HSA D1 shuffle S7-31 | | E | | | | | | | | | | |
| HSA D1 shuffle S7-32 | | E | | | | D | | | | | | |
| HSA D1 shuffle S7-33 | | E | | | | | | | | | | |
| Position | | 137 | 142 | 149 | 153 | 156 | 157 | 159 | 160 | 162 | 165 | |
| wt | | K | I | F | E | F | F | K | R | K | F | |
| HSA D1 shuffle S7-1 | | | | S | | | | | | R | | |
| HSA D1 shuffle S7-2 | | | | S | | | | | | | | |
| HSA D1 shuffle S7-4 | | | | | | | | | | R | | |
| HSA D1 shuffle S7-5 | | | | S | | | | | | | | |
| HSA D1 shuffle S7-6 | | | | S | | | | | | E | | |
| HSA D1 shuffle S7-8 | | | | S | | | | | | | | |
| HSA D1 shuffle S7-9 | | | | S | | | | | | | | |
| HSA D1 shuffle S7-13 | | | | S | | S | L | | | | | |
| HSA D1 shuffle S7-14 | | | | S | | | | | | | | |
| HSA D1 shuffle S7-17 | | | V | S | | | | | | | | |
| HSA D1 shuffle S7-20 | | | | S | | | | | | | | |
| HSA D1 shuffle S7-22 | | | | S | | | | | | | | |
| HSA D1 shuffle S7-23 | | | | S | | | | | | | | |
| HSA D1 shuffle S7-26 | | | | S | G | | | R | | | | |
| HSA D1 shuffle S7-27 | | | | S | | | | | | | | |
| HSA D1 shuffle S7-28 | | | | S | | | | | | | L | |
| HSA D1 shuffle S7-29 | | R | T | S | | | | | | | | |
| HSA D1 shuffle S7-30 | | | | S | | | | | | | | |
| HSA D1 shuffle S7-31 | | | | S | | | | | | | | |
| HSA D1 shuffle S7-32 | | | | S | | | | | G | | | |
| HSA D1 shuffle S7-33 | | | | S | | S | L | | | | | |
| Position | | 170 | 174 | 183 | 184 | 188 | 190 | 192 | 196 | 198 | 199 | |
| wt | | Q | K | D | E | E | K | S | Q | L | K | |
| HSA D1 shuffle S7-1 | | | | | | | E | P | R | | E | |
| HSA D1 shuffle S7-2 | | | | | | | E | P | | | | |
| HSA D1 shuffle S7-4 | | | | | | G | E | | | P | R | |
| HSA D1 shuffle S7-5 | | | | | | | E | P | | | | |
| HSA D1 shuffle S7-6 | | | | | | | E | P | | | | |
| HSA D1 shuffle S7-8 | | | | | | | E | P | R | | E | |
| HSA D1 shuffle S7-9 | | R | | | G | | E | P | | | | |
| HSA D1 shuffle S7-13 | | | | G | | | E | P | | | | |
| HSA D1 shuffle S7-14 | | | | | | | E | P | R | | E | |
| HSA D1 shuffle S7-17 | | | | | | | E | P | | | | |
| HSA D1 shuffle S7-20 | | | | | | | E | P | | | | |
| HSA D1 shuffle S7-22 | | | | | | | E | P | R | | E | |
| HSA D1 shuffle S7-23 | | | | | | | E | P | | | | |
| HSA D1 shuffle S7-26 | | R | R | | | | E | P | R | E | | |
| HSA D1 shuffle S7-27 | | | R | | | | E | P | R | | E | |
| HSA D1 shuffle S7-28 | | | | | | | E | P | | | | |
| HSA D1 shuffle S7-29 | | | | | | | E | P | | | | |
| HSA D1 shuffle S7-30 | | | | | | | E | P | | | | |
| HSA D1 shuffle S7-31 | | | | | | | E | P | | | | |
| HSA D1 shuffle S7-32 | | | | | | | E | P | | | | |
| HSA D1 shuffle S7-33 | | | | | | | E | P | R | | E | |

HSA D1 Shuffle S7-2 and HSA D1 Shuffle S7-8 were repeated 13 and 23 times, respectively

### Example 6: Binding of Selected Individual Clones to FcRn

Single clones were picked, expressed on the surface of yeast, and assayed for binding to FcRn using the methods described above. Individual clones were incubated with varying concentrations of FcRn at pH 5.0, pH 5.5, pH 6.0, pH 6.5, pH 7.0, or pH 7.5 to establish a dose-response at different pH's. The binding EC₅₀ of single clones were calculated by plotting the mean fluorescence intensity (MFI) values versus FcRn concentration as shown in Table 6 (nd=not determined due to insufficient binding). Table 7 shows binding MFI of single clones at saturation, which is a relative function of off-rate (nd=not determined due to insufficient binding).

TABLE 6

| | pH 5.0 | pH 5.5 | pH 6.0 | pH 6.5 | pH 7.0 | pH 7.5 |
|---|---|---|---|---|---|---|
| | EC₅₀ (M) | EC₅₀ (M) | EC₅₀ (M) | EC_{5 0}(M) | EC₅₀ (M) | EC₅₀ (M) |
| S7-1 | 7.60E−09 | 8.90E−09 | 2.10E−08 | 2.20E−07 | nd | nd |
| S7-2 | 5.50E−09 | 5.50E−09 | 1.10E−08 | 1.80E−07 | nd | nd |
| S7-3 | 6.40E−09 | 7.10E−09 | 1.40E−08 | 9.20E−08 | nd | nd |
| S7-4 | 5.30E−09 | 6.10E−09 | 1.50E−08 | nd | nd | nd |
| S7-5 | 4.90E−09 | 6.70E−09 | 3.40E−08 | nd | nd | nd |
| S7-6 | 5.80E−09 | 7.10E−09 | 1.50E−08 | 1.10E−07 | nd | nd |
| S6high-1 | 5.80E−09 | 6.50E−09 | 7.20E−09 | 1.50E−08 | 6.20E−08 | 1.30E−07 |
| S6high-2 | 6.50E−09 | 5.70E−09 | 6.20E−09 | 1.00E−08 | 1.30E−07 | 4.10E−07 |
| wtHSA | nd | nd | nd | nd | nd | nd |

TABLE 7

| | pH 5.0 | pH 5.5 | pH 6.0 | pH 6.5 | pH 7.0 | pH 7.5 |
|---|---|---|---|---|---|---|
| | max | max | max | max | max | max |
| | MFI | MFI | MFI | MFI | MFI | MFI |
| S7-1 | 11402 | 11131 | 10178 | 6911 | nd | nd |
| S7-2 | 11354 | 10910 | 10205 | 6225 | nd | nd |
| S7-3 | 12642 | 12618 | 12070 | 7563 | nd | nd |
| S7-4 | 9986 | 9427 | 8704 | 7472 | nd | nd |
| S7-5 | 12221 | 11752 | 9842 | 3577 | nd | nd |
| S7-6 | 10777 | 10572 | 9666 | 4729 | nd | nd |
| S6high-1 | 8311 | 8208 | 7970 | 7304 | 8823 | 5006 |
| S6high-2 | 11584 | 10591 | 10275 | 7810 | 9944 | 3982 |
| HA13 | 16231 | 16549 | 16830 | 15401 | 19371 | 13195 |
| wtHSA | nd | nd | nd | nd | nd | nd |

Additionally, the individual clones are expressed recombinantly in mammalian cells, E. coli, or yeast and purified by using chromatography or other protein biochemistry techniques known in the art. Soluble recombinant albumin clones are assayed for binding to FcRn by ELISA, Biacore, or related assay at various pH levels.

### Example 7: In Vivo Half-Life of Engineered Albumins

The half-life of particular engineered albumins is measured according to methods known in the art. Engineered albumins are expressed in mammalian cells, E. coli, or yeast and purified by chromatography or other protein biochemistry techniques known in the art. In some embodiments, the expression constructs contain C-terminal or N-terminal epitope tags (e.g. polyhistidine, c-Myc, FLAG, HA, V5) to simplify detection. Single-dose or multidose pharmacokinetic (PK) profiles are obtained by intravenously (IV) or intraperitoneally (IP) administering the engineered albumin to a subject animal (e.g. mouse, rat, cynomolgus monkey). Blood is drawn at appropriate time points (e.g. 5 minutes, 15 minutes, and 1, 2, 4, 8, 24, 48, 72, 96, 120 hours or longer times post-dosing). Plasma concentrations of albumins are determined using Enzyme Linked Immunosorbant Assays (ELISA) or other assays known in the art. In some embodiments, immunoassays such as ELISA utilize a human-specific anti-albumin antibody (e.g., R&D Systems, Minneapolis, Minn., Cat. No. MAB1455) or an anti-epitope tag antibody (e.g. Thermo Fisher Scientific, Cat. No. A-21281). The half-life can be measured in a wild-type mouse or a genetically modified mouse such as a human FcRn knock-in or a serum albumin knock-out.

### Example 8: Kinetics of Engineered Albumins

The selected FcRn-enhanced albumin clones are expressed in mammalian cells, E. coli, or yeast, and purified by chromatography or other protein biochemistry techniques known in the art. In some embodiments, the affinity of each variant for FcRn is determined by surface plasmon resonance using a BIAcoreT200 or similar instrument according to standard methods. The assay is conducted at pH 5.5, pH 7.4, or an intermediate pH. In other embodiments, a Biacore Series S CMS sensor chips (GE Healthcare, Little Chalfont, UK) are immobilized with monoclonal mouse anti-biotin antibody. The biotinylated FcRn is then captured on to the chip. Serial dilutions of each variant are injected at a flow rate of 30 μl/min. In other experiments, the engineered albumin is captured with anti-albumin or an antibody against an epitope tag (such as poly-histidine) coated on the CMS chip, and antigen is flowed over the chip. Each sample is analyzed, for example, with 3-minute association and 10-minute dissociation. After each injection the chip is regenerated using 3 M MgCl₂ or another appropriate buffer. Binding response is corrected by subtracting the response units (RUs) from a flow cell capturing an irrelevant IgG at similar density. A 1:1 Languir model of simultaneous fitting of kₒₙ and k_{off} is used for kinetics analysis.

### Exemplary Sequences:

Exemplary sequences are provided as follows:

Yeast codon-optimized full length Human Serum
Albumin gene DNA sequence
SEQ ID NO: 1
GACGCTCATAAATCTGAAGTAGCACACAGATTTAAAGACCTAGGTGAAGAGAATTTCAAAGC
CTTGGTTTTAATTGCATTCGCTCAGTATTTGCAACAATGTCCGTTTGAAGACCATGTTAAAC
TAGTTAATGAGGTCACCGAGTTTGCAAAAACATGTGTCGCTGACGAATCCGCTGAGAATTGC
GACAAATCATTGCATACTTTATTCGGCGATAAGTTATGCACTGTTGCTACTCTACGTGAAAC
ATATGGTGAAATGGCCGATTGTTGCGCCAAACAAGAACCTGAGAGAAATGAATGCTTTTTAC
AACATAAAGATGATAACCCAAATTTACCTAGGTTAGTTAGACCGGAGGTTGACGTTATGTGT
ACCGCATTTCACGACAATGAAGAGACGTTCCTGAAGAAGTATTTATATGAAATCGCAAGAAG
ACATCCTTATTTTTATGCACCAGAGTTGTTATTCTTCGCTAAAAGATATAAGGCTGCATTCA
CTGAGTGTTGCCAAGCAGCAGATAAGGCAGCATGTCTGTTACCAAAGTTAGATGAGTTACGT
GACGAAGGGAAGGCGTCATCTGCTAAGCAACGTCTGAAATGTGCAAGCTTACAAAAGTTTGG
AGAAAGGGCCTTTAAGGCTTGGGCTGTGGCGAGGTTAAGTCAGAGATTCCCAAAAGCCGAAT
TTGCTGAGGTGAGCAAATTGGTAACCGACTTGACAAAAGTTCATACAGAATGTTGTCACGGT
GATCTATTGGAGTGTGCAGACGATAGAGCGGACTTGGCCAAGTATATTTGTGAGAATCAAGA
TAGTATCAGCTCTAAATTAAAGGAATGCTGTGAAAAACCATTATTGGAAAAGTCTCACTGTA
TTGCTGAAGTAGAAAACGATGAGATGCCAGCCGATCTTCCCTCTCTAGCAGCTGATTTTGTC
GAGTCTAAGGACGTTTGCAAGAATTATGCCGAAGCTAAAGACGTTTTTTTGGGGATGTTCTT
ATACGAATATGCGCGCAGGCATCCAGACTACTCCGTGGTACTATTACTGAGATTGGCGAAGA
CGTACGAAACGACTCTGGAAAAATGCTGTGCGGCGGCCGATCCACATGAGTGTTACGCTAAG
GTATTCGATGAGTTTAAACCATTAGTAGAAGAGCCACAAAATTTAATCAAGCAAAATTGTGA
GCTGTTTGAACAATTGGGTGAATACAAATTCCAGAACGCCCTTTTGGTTAGATACACAAAGA
AAGTGCCCCAGGTATCTACTCCAACTCTTGTTGAAGTGTCCAGAAACTTAGGCAAAGTTGGG
AGTAAATGTTGTAAGCACCCTGAAGCAAAGAGAATGCCTTGTGCGGAAGATTATTTATCCGT
TGTGCTAAATCAGTTGTGTGTTTTGCATGAGAAAACCCCCGTATCAGATAGAGTGACCAAGT
GTTGCACGGAGTCACTGGTTAATAGGAGGCCATGTTTTTCAGCATTAGAGGTTGATGAGACA
TACGTCCCCAAAGAGTTTAACGCGGAGACATTCACGTTCCACGCAGACATCTGCACCCTATC
TGAAAAGGAAAGACAAATAAAAAAGCAGACCGCTCTTGTAGAGTTAGTAAAACATAAGCCTA
AGGCAACGAAGGAACAGTTGAAAGCAGTAATGGACGATTTTGCCGCATTTGTCGAGAAATGT
TGTAAAGCAGATGATAAAGAGACGTGCTTTGCCGAAGAAGGAAAAAAGTTAGTTGCGGCTAG
TCAAGCAGCGTTAGGGTTA
Mature Human Serum Albumin starting at Domain 1
SEQ ID NO: 2
DAHKSEVAHRFKDLGEENFKALVLIAFAQYLQQCPFEDHVKLVNEVTEFAKTCVADESAENC
DKSLHTLFGDKLCTVATLRETYGEMADCCAKQEPERNECFLQHKDDNPNLPRLVRPEVDVMC
TAFHDNEETFLKKYLYEIARRHPYFYAPELLFFAKRYKAAFTECCQAADKAACLLPKLDELR
DEGKASSAKQRLKCASLQKFGERAFKAWAVARLSQRFPKAEFAEVSKLVTDLTKVHTECCHG
DLLECADDRADLAKYICENQDSISSKLKECCEKPLLEKSHCIAEVENDEMPADLPSLAADFV
ESKDVCKNYAEAKDVFLGMFLYEYARRHPDYSVVLLLRLAKTYETTLEKCCAAADPHECYAK
VFDEFKPLVEEPQNLIKQNCELFEQLGEYKFQNALLVRYTKKVPQVSTPTLVEVSRNLGKVG
SKCCKHPEAKRMPCAEDYLSVVLNQLCVLHEKTPVSDRVTKCCTESLVNRRPCFSALEVDET
YVPKEFNAETFTFHADICTLSEKERQIKKQTALVELVKHKPKATKEQLKAVMDDFAAFVEKC
CKADDKETCFAEEGKKLVAASQAALGL
Forward PCR Primer
SEQ ID NO: 3
CTAGTGGTGGAGGAGGCTCTGGTGGAGGCGGTAGCGGAGGCGGAGGGTCGGCTAGC
Reverse PCR Primer
SEQ ID NO: 4
TCGCCACAGCCCAAGCCTTAAAGGCCCTTTCTCCAAACTTTTGTAAGCTTGCACA
Human FcRn alpha subunit
SEQ ID NO: 5
MGVPRPQPWALGLLLFLLPGSLGAESHLSLLYHLTAVSSPAPGTPAFWVSGWLGPQQYLS
YNSLRGEAEPCGAWVWENQVSWYWEKETTDLRIKEKLFLEAFKALGGKGPYTLQGLLGCE
LGPDNTSVPTAKFALNGEEFMNFDLKQGTWGGDWPEALAISQRWQQQDKAANKELTFLLF
SCPHRLREHLERGRGNLEWKEPPSMRLKARPSSPGFSVLTCSAFSFYPPELQLRFLRNGL
AAGTGQGDFGPNSDGSFHASSSLTVKSGDEHHYCCIVQHAGLAQPLRVELESPAKSSVLV
VGIVIGVLLLTAAAVGGALLWRRMRSGLPAPWISLRGDDTGVLLPTPGEAQDADLKDVNV
IPATA
Mature human serum albumin domain 1
SEQ ID NO: 6
DAHKSEVAHRFKDLGEENFKALVLIAFAQYLQQCPFEDHVKLVNEVTEFAKTCVADESAENC
DKSLHTLFGDKLCTVATLRETYGEMADCCAKQEPERNECFLQHKDDNPNLPRLVRPEVDVMC
TAFHDNEETFLKKYLYEIARRHPYFYAPELLFFAKRYKAAFTECCQAADKAACLLPKLDELR
DEGKASSAKQRLK
Mature human serum albumin domain 2
SEQ ID NO: 7
CASLQKFGERAFKAWAVARLSQRFPKAEFAEVSKLVTDLTKVHTECCHGDLLECADDRADLA
KYICENQDSISSKLKECCEKPLLEKSHCIAEVENDEMPADLPSLAADFVESKDVCKNYAEAK
DVFLGMFLYEYARRHPDYSVVLLLRLAKTYETTLEKCCAAADPHECYAKVFDEFKP
Mature human serum albumin domain 3
SEQ ID NO: 8
LVEEPQNLIKQNCELFEQLGEYKFQNALLVRYTKKVPQVSTPTLVEVSRNLGKVGSKCCKHP
EAKRMPCAEDYLSVVLNQLCVLHEKTPVSDRVTKCCTESLVNRRPCFSALEVDETYVPKEFN
AETFTFHADICTLSEKERQIKKQTALVELVKHKPKATKEQLKAVMDDFAAFVEKCCKADDKE
TCFAEEGKKLVAASQAALGL
HSA Clone 7-1
SEQ ID NO: 9
DAHKSEVAHRFKDLGEENFKALVLIAFAQYLQQCPFEDHVKLVNEVTEFAKTCVADESAENC
DESLHTLFGDKLCTVATLRETYGEMVDCCAEQEPERNECFLQHKDDNPNLPRLVRPEVDVMC
TAFHDNEETFLKKYLYEIARRHPYSYAPELLFFAKRYKAAFTECCQAADKAACLLPKLDELR
DEGEAPSAKRRLECASLQKFGERAFKAWAVARLSQRFPKAEFAEVSKLVTDLTKVHTECCHG
DLLECADDRADLAKYICENQDSISSKLKECCEKPLLEKSHCIAEVENDEMPADLPSLAADFV
ESKDVCKNYAEAKDVFLGMFLYEYARRHPDYSVVLLLRLAKTYETTLEKCCAAADPHECYAK
VFDEFKPLVEEPQNLIKQNCELFEQLGEYKFQNALLVRYTKKVPQVSTPTLVEVSRNLGKVG
SKCCKHPEAKRMPCAEDYLSVVLNQLCVLHEKTPVSDRVTKCCTESLVNRRPCFSALEVDET
YVPKEFNAETFTFHADICTLSEKERQIKKQTALVELVKHKPKATKEQLKAVMDDFAAFVEKC
CKADDKETCFAEEGKKLVAASQAALGL
HSA clone 7-2
SEQ ID NO: 10
DAHKSEVAHRFKDLGEENLEALVLIAFAQYLQQCPFEDHVKLVNEVTEFAKTCVADESAENC
DKSLHTLFGDKLCAVTTLRETYGEMADCCAEQEPERNECFLQHRDDNPNLPRLVRPEVDVMC
TAFHDNEETFLKKYLYEIARRHPYSYAPELLFFAKRYKAAFTECCQAADKAACLLPKLDELR
DEGEAPSAKQRLKCASLQKFGERAFKAWAVARLSQRFPKAEFAEVSKLVTDLTKVHTECCHG
DLLECADDRADLAKYICENQDSISSKLKECCEKPLLEKSHCIAEVENDEMPADLPSLAADFV
ESKDVCKNYAEAKDVFLGMFLYEYARRHPDYSVVLLLRLAKTYETTLEKCCAAADPHECYAK
VFDEFKPLVEEPQNLIKQNCELFEQLGEYKFQNALLVRYTKKVPQVSTPTLVEVSRNLGKVG
SKCCKHPEAKRMPCAEDYLSVVLNQLCVLHEKTPVSDRVTKCCTESLVNRRPCFSALEVDET
YVPKEFNAETFTFHADICTLSEKERQIKKQTALVELVKHKPKATKEQLKAVMDDFAAFVEKC
CKADDKETCFAEEGKKLVAASQAALGL
HSA clone 7-3
SEQ ID NO: 11
DAHKSEVAHGFKDLGEENFKALVLIAFAQYLQQCPFEDHVKLVNEVTEFAKTCVADESAENC
DKSLHTLFGDKLCTVATLRETYGEMVDCCAEQEPERNECFLQHKDDNPNLPRLVRPEVDVMC
TAFHDDEETFLKKYLYEIARRHPYSYAPELLFFAKRYKAAFTECCQAADRAACLLPKLDELR
DEGEAPSAKRRLECASLQKFGERAFKAWAVARLSQRFPKAEFAEVSKLVTDLTKVHTECCHG
DLLECADDRADLAKYICENQDSISSKLKECCEKPLLEKSHCIAEVENDEMPADLPSLAADFV
ESKDVCKNYAEAKDVFLGMFLYEYARRHPDYSVVLLLRLAKTYETTLEKCCAAADPHECYAK
VFDEFKPLVEEPQNLIKQNCELFEQLGEYKFQNALLVRYTKKVPQVSTPTLVEVSRNLGKVG
SKCCKHPEAKRMPCAEDYLSVVLNQLCVLHEKTPVSDRVTKCCTESLVNRRPCFSALEVDET
YVPKEFNAETFTFHADICTLSEKERQIKKQTALVELVKHKPKATKEQLKAVMDDFAAFVEKC
CKADDKETCFAEEGKKLVAASQAALGL
HSA clone 7-4
SEQ ID NO: 12
DAHKSEVAHGFKDLGEENFKALVLIAFAQYLQQCPFEDHVKLVNEVTEFAKTCVADESAENC
DKSLHTLFGDKLCTVATLRETYGEMVDCCAEQEPERNECFLQHRDDNPDLPRLVRPGVDVMC
TAFHDNEETFLKKYLYEIARRHPYSYAPELLFFAKRYKAAFTECCQAADKAACLLPKLDELR
DEGEAPSAKQRLKCASLQKFGERAFKAWAVARLSQRFPKAEFAEVSKLVTDLTKVHTECCHG
DLLECADDRADLAKYICENQDSISSKLKECCEKPLLEKSHCIAEVENDEMPADLPSLAADFV
ESKDVCKNYAEAKDVFLGMFLYEYARRHPDYSVVLLLRLAKTYETTLEKCCAAADPHECYAK
VFDEFKPLVEEPQNLIKQNCELFEQLGEYKFQNALLVRYTKKVPQVSTPTLVEVSRNLGKVG
SKCCKHPEAKRMPCAEDYLSVVLNQLCVLHEKTPVSDRVTKCCTESLVNRRPCFSALEVDET
YVPKEFNAETFTFHADICTLSEKERQIKKQTALVELVKHKPKATKEQLKAVMDDFAAFVEKC
CKADDKETCFAEEGKKLVAASQAALGL
HSA clone 7-5
SEQ ID NO: 13
DAHKSEVAHRFKDLGEENFKALVLIAFAQYLQQCPFEDHVKLVNEVTEFAKTCVADESAENC
DKSLHTLFGDKLCTVATLRETYGEMVDCCAEQEPERNECFLQHKDDNPDLPRLVRPEVDVMC
TAFHDNEETFLKKYLYEIARRHPYSYAPELLFFAKGYKAAFTECCQAADKAACLLPKLDELR
DEGEAPSAKQRLKCASLQKFGERAFKAWAVARLSQRFPKAEFAEVSKLVTDLTKVHTECCHG
DLLECADDRADLAKYICENQDSISSKLKECCEKPLLEKSHCIAEVENDEMPADLPSLAADFV
ESKDVCKNYAEAKDVFLGMFLYEYARRHPDYSVVLLLRLAKTYETTLEKCCAAADPHECYAK
VFDEFKPLVEEPQNLIKQNCELFEQLGEYKFQNALLVRYTKKVPQVSTPTLVEVSRNLGKVG
SKCCKHPEAKRMPCAEDYLSVVLNQLCVLHEKTPVSDRVTKCCTESLVNRRPCFSALEVDET
YVPKEFNAETFTFHADICTLSEKERQIKKQTALVELVKHKPKATKEQLKAVMDDFAAFVEKC
CKADDKETCFAEEGKKLVAASQAALGL
HSA clone 7-6
SEQ ID NO: 14
DAHKPEVAHRFKDLGEENFKALVLIAFAQYLQQCPFEDHVKLVNEVTEFAKTCVADESAENC
DKSLHTLFGDKLCTVATLRETYGEMVDCCAEQEPERNECFLQHKDDNPNLPRLVRPEVDVMC
TAFHDNEETFLKKYLYEIARRHPYSYAPELLFFAKRYKAAFTECCQAADKAACLLPKLDELR
DEGEAPSAKRRLECASLQKFGERAFKAWAVARLSQRFPKAEFAEVSKLVTDLTKVHTECCHG
DLLECADDRADLAKYICENQDSISSKLKECCEKPLLEKSHCIAEVENDEMPADLPSLAADFV
ESKDVCKNYAEAKDVFLGMFLYEYARRHPDYSVVLLLRLAKTYETTLEKCCAAADPHECYAK
VFDEFKPLVEEPQNLIKQNCELFEQLGEYKFQNALLVRYTKKVPQVSTPTLVEVSRNLGKVG
SKCCKHPEAKRMPCAEDYLSVVLNQLCVLHEKTPVSDRVTKCCTESLVNRRPCFSALEVDET
YVPKEFNAETFTFHADICTLSEKERQIKKQTALVELVKHKPKATKEQLKAVMDDFAAFVEKC
CKADDKETCFAEEGKKLVAASQAALGL
HSA clone 7-7
SEQ ID NO: 15
DAHKSEVAHRFKDLGEENFKALVLIAFAQYLQQCPFEDHVKLVNEVTEFAKTCVADESAENC
DKSLHTLFGDKLCTVATLRETYGEMADCCAEQEPERNECFLQHKDDNPNLPRLVRPEVDVMC
TAFHDNEETFLKKYLYEIARRHPYSYAPELLFFAKRYKAAFTECCQAADKAACLLPKLDELR
DEGEAPSAKQRLKCASLQKFGERAFKAWAVARLSQRFPKAEFAEVSKLVTDLTKVHTECCHG
DLLECADDRADLAKYICENQDSISSKLKECCEKPLLEKSHCIAEVENDEMPADLPSLAADFV
ESKDVCKNYAEAKDVFLGMFLYEYARRHPDYSVVLLLRLAKTYETTLEKCCAAADPHECYAK
VFDEFKPLVEEPQNLIKQNCELFEQLGEYKFQNALLVRYTKKVPQVSTPTLVEVSRNLGKVG
SKCCKHPEAKRMPCAEDYLSVVLNQLCVLHEKTPVSDRVTKCCTESLVNRRPCFSALEVDET
YVPKEFNAETFTFHADICTLSEKERQIKKQTALVELVKHKPKATKEQLKAVMDDFAAFVEKC
CKADDKETCFAEEGKKLVAASQAALGL
HSA clone 7-8
SEQ ID NO: 16
DAHKSEVAHRFKDLGEENFKALVLIAFAQYLQQCPFEDHVKLVNEVTEFAKTCVADESAENC
DKSLHTLFGDKLCTVATLRETYGEMADCCAEQEPERNECFLQHKDDNPNLPRLVRPEVDVMC
TAFHDNEETFLKKYLYEIARRHPYSYAPELLFFAKRYKAAFTECCQAADKAACLLPKLDELR
DEGEAPSAKRRLECASLQKFGERAFKAWAVARLSQRFPKAEFAEVSKLVTDLTKVHTECCHG
DLLECADDRADLAKYICENQDSISSKLKECCEKPLLEKSHCIAEVENDEMPADLPSLAADFV
ESKDVCKNYAEAKDVFLGMFLYEYARRHPDYSVVLLLRLAKTYETTLEKCCAAADPHECYAK
VFDEFKPLVEEPQNLIKQNCELFEQLGEYKFQNALLVRYTKKVPQVSTPTLVEVSRNLGKVG
SKCCKHPEAKRMPCAEDYLSVVLNQLCVLHEKTPVSDRVTKCCTESLVNRRPCFSALEVDET
YVPKEFNAETFTFHADICTLSEKERQIKKQTALVELVKHKPKATKEQLKAVMDDFAAFVEKC
CKADDKETCFAEEGKKLVAASQAALGL
HSA clone 7-9
SEQ ID NO: 17
DAHKSEVAHRFKDLGEENFKALVLIAFAQYLQQCPFEDHVKLVNEVTEFAKTCVADESAENC
DKSLHTLFGDKLCTVATLRETYGEMVDCCAEQEPERNECFLQHKDDNPNLPRLVRPEVDVMC
TAFHDNEETFLKKYLYEIARRHPYSYAPELLFFAKRYKAAFTECCQAADKAACLLPKLDELR
DEGEAPSAKRRLECASLQKFGERAFKAWAVARLSQRFPKAEFAEVSKLVTDLTKVHTECCHG
DLLECADDRADLAKYICENQDSISSKLKECCEKPLLEKSHCIAEVENDEMPADLPSLAADFV
ESKDVCKNYAEAKDVFLGMFLYEYARRHPDYSVVLLLRLAKTYETTLEKCCAAADPHECYAK
VFDEFKPLVEEPQNLIKQNCELFEQLGEYKFQNALLVRYTKKVPQVSTPTLVEVSRNLGKVG
SKCCKHPEAKRMPCAEDYLSVVLNQLCVLHEKTPVSDRVTKCCTESLVNRRPCFSALEVDET
YVPKEFNAETFTFHADICTLSEKERQIKKQTALVELVKHKPKATKEQLKAVMDDFAAFVEKC
CKADDKETCFAEEGKKLVAASQAALGL
β₂ Microglobulin
SEQ ID NO: 18
MSRSVALAVLALLSLSGLEAIQRTPKIQVYSRHPAENGKSNFLNCYVSGFHPSDIEVDLLKN
GERIEKVEHSDLSFSKDWSFYLLYYTEFTPTEKDEYACRVNHVTLSQPKIVKWDRDM

All publications and patent documents disclosed or referred to herein are incorporated by reference in their entirety. The foregoing description has been presented only for purposes of illustration and description. This description is not intended to limit the invention to the precise form disclosed. It is intended that the scope of the invention be defined by the claims appended hereto.

What is claimed:

1. An albumin variant polypeptide, comprising at least one amino acid substitution in serum albumin domain 1, and wherein said at least one amino acid substitution enhances the specific binding between said albumin variant polypeptide and an FcRn polypeptide.

2. An albumin variant polypeptide, comprising at least one amino acid substitution in a structural region that does not directly interact with an FcRn polypeptide, wherein said at least one amino acid substitution enhances the specific binding between said albumin variant polypeptide and said FcRn polypeptide.

3. An albumin variant polypeptide, comprising at least one amino acid substitution in SEQ ID NO:2, wherein said substitution is at a position selected from the group consisting of 1, 3, 5, 6, 7, 10, 11, 12, 13, 14, 15, 16, 18, 19, 20, 24, 25, 27, 30, 31, 32, 34, 37, 40, 41, 43, 44, 50, 51, 56, 57, 60, 61, 64, 67, 68, 76, 77, 78, 87, 88, 89, 90, 92, 93, 94, 95, 99, 101, 106, 109, 111, 112, 116, 119, 120, 130, 136, 137, 138, 142, 145, 149, 152, 153, 156, 157, 159, 160, 162, 163, 165, 170, 171, 174, 183, 184, 188, 189, 190, 191, 192, 193, 194, 196, 198, and 199, and wherein said albumin variant polypeptide specifically binds FcRn.

4. The albumin variant polypeptide of claim 3, wherein said at least one amino acid substitution is selected from the group consisting of D1G, H3Y, S5P, R10G, K12R, D13G, N18D, K20E, I25V, F27L, F27S, C34R, A50V, K51R, E60G, K64E, T76A, V77I, A78T, C90W, K93E, E95G, C101W, K106R, N111D, V116A, E119G, V120A, N130D, K136E, K137R, I142V, F149S, E153G, F156S, F157L, K159R, R160G, K162R, K162E, F165L, Q170R, K174R, D183G, E184G, E188G, K190E, S192P, L198P, and K199R

5. The albumin variant polypeptide of claim 3, wherein said at least one amino acid substitution is selected from the group consisting of D1G, S5P, E6G, V7A, R10G, F11S, K12R, D13G, L14P, G15D, E16G, N18D, F19L, K20E, K20R, L24S, I25V, F27C, F27L, Y30H, Q32R, E37K, V40A, K41E, V43I, N44E, A50V, K51R, D56G, D56T, E57G, N61S, K64E, T76A, A78T, A88V, A92T, K93E, Q94R, E95G, N99S, K106R, N109D, N111D, N111E, E119G, N130D, K136E, Y138H, I142V, I142T, R145G, P152S, F156S, K162R, K162E, A163T, A171V, E184G, E188G, G189R, K190E, A191V, S192P, A194T, Q196R, L198P, K199R, and K199E.

6. The albumin variant polypeptide of claim 3, wherein said at least one amino acid substitution is selected from the group consisting of S5P, D13G, N18D, K20E, L24S, K41E, K41R, V43A, D56G, H67Y, T68A, M87I, K93E, E95G, N99D, K106R, N111S, L112S, I142T, F149S, P152S, P152L, R160G, F165L, K190E, K190R, L198P, K199R, and K199E.

7. The albumin variant polypeptide of claim 3, wherein said at least one amino acid substitution is selected from the group consisting of Y30C, L31S, Q32R, K41E, A50T, K51R, T76A, D89G, N130D, K162E, E188G, K190E, and S192P.

8. The albumin variant polypeptide of any of the proceeding claims, comprising a plurality of said amino acid substitutions.

9. The albumin variant polypeptide of claim 3, wherein said polypeptide comprises the amino acid substitution F19L.

10. The albumin variant polypeptide of claim 3, wherein said polypeptide comprises the amino acid substitution L24S.

11. The albumin variant polypeptide of claim 3, wherein said polypeptide comprises the amino acid substitution A88V.

12. The albumin variant polypeptide of claim 3, wherein said polypeptide comprises the amino acid substitution K93E.

13. The albumin variant polypeptide of claim 3, wherein said polypeptide comprises the amino acid substitution F149S.

14. The albumin variant polypeptide of claim 1, wherein said polypeptide comprises the amino acid substitution K190E.

15. The albumin variant polypeptide of claim 3, wherein said polypeptide comprises the amino acid substitution S192P.

16. The albumin variant polypeptide of claim 3, wherein said polypeptide comprises the amino acid substitution Q196R.

17. The albumin variant polypeptide of claim 3, wherein said polypeptide comprises the amino acid substitution K199E.

18. The albumin variant polypeptide of claim 3, wherein said polypeptide has a sequence set forth in any one of SEQ ID NOS:9-16.

19. The albumin variant of polypeptide of claim 3, wherein said polypeptide has any one or more of the amino acid variations found in SEQ ID NOS:9-17 when compared to SEQ ID NO:2.

20. The albumin variant polypeptide of any of the proceeding claims, wherein said albumin is derived from a human albumin.

21. The albumin variant polypeptide of any of the proceeding claims, wherein said polypeptide specifically binds to an FcRn protein with increased affinity when compared to the serum albumin of SEQ ID NO:2.

22. The albumin variant polypeptide of any of the proceeding claims, wherein said polypeptide specifically binds to an FcRn protein with increased affinity at a pH of about 5.5 when compared to the serum albumin of SEQ ID NO:2.

23. The albumin variant polypeptide of any of the proceeding claims, wherein said polypeptide specifically binds to an FcRn protein with an increased affinity at an acidic pH when compared to its affinity at a neutral pH.

24. The albumin variant polypeptide of any of the proceeding claims, wherein said polypeptide specifically binds to an FcRn protein with a greater affinity at a pH of about 5.5 when compared to its affinity at a pH of about 7.4.

25. The albumin variant polypeptide of any of the proceeding claims, wherein said polypeptide has a first binding affinity for said FcRn at a pH of about 5.5 and a second binding affinity for said FcRn at a pH of about 7.4, wherein
a. said first binding affinity is between 10 and 20 fold higher than said second binding affinity;
b. said first binding affinity is between 21 and 30 fold higher than said second binding affinity;
c. said first binding affinity is between 31 and 40 fold higher than said second binding affinity;
d. said first binding affinity is between 41 and 50 fold higher than said second binding affinity;
e. said first binding affinity is between 51 and 60 fold higher than said second binding affinity;
f. said first binding affinity is between 61 and 70 fold higher than said second binding affinity;
g. said first binding affinity is between 71 and 80 fold higher than said second binding affinity;
h. said first binding affinity is between 81 and 90 fold higher than said second binding affinity;
i. said first binding affinity is between 91 and 100 fold higher than said second binding affinity;
j. said first binding affinity is between 101 and 1,000 fold higher than said second binding affinity;
k. said first binding affinity is between 1,001 and 10,000 fold higher than said second binding affinity; or
l. said first binding affinity is between 10,001 and 100,000 fold higher than said second binding affinity.

26. The albumin variant polypeptide of any of the proceeding claims, wherein said polypeptide specifically binds to an FcRn protein at a pH of about 5.5 and does not specifically bind to said FcRn at a pH of about 7.4.

27. The albumin variant polypeptide of any of the proceeding claims, wherein said polypeptide specifically binds to an FcRn protein with a Kd of less than about 1 μM, less than about 100 nM, or less than about 10 nM at a pH of about 5.5.

28. The albumin variant polypeptide of claim 21, wherein said polypeptide specifically binds to an FcRn protein with a Kd of more than about 1 μM, less than about 1 μM, or less than about 100 nM at a pH of about 7.4.

29. The albumin variant polypeptide of either one of claims 27 or 28, wherein said Kd is measured using an enzyme linked immunosorbant assay (ELISA), surface plasmon resonance (SPR) binding assay, or cell surface binding assay.

30. The albumin variant polypeptide of any of the proceeding claims, further comprising one or more additional amino acid modifications.

31. An albumin variant polypeptide, wherein at least a portion of said polypeptide comprises an amino acid sequence having at least a 90% sequence identity to SEQ ID NO:2 and wherein said albumin variant polypeptide specifically binds to FcRn at a pH of about 5.5.

32. The albumin variant polypeptide of claim 25, comprising an amino acid sequence having at least a 90% sequence identity to amino acid numbers 1-199 of SEQ ID NO:2 and wherein said albumin variant polypeptide specifically binds to FcRn at a pH of about 5.5.

33. An albumin variant polypeptide, wherein at least a portion of said polypeptide comprises an amino acid sequence having at least a 90% sequence identity to SEQ ID NO:9, SEQ ID NO:10, SEQ ID NO:11, SEQ ID NO:12, SEQ ID NO:13, SEQ ID NO:14, SEQ ID NO:15, SEQ ID NO:16, or SEQ ID NO:17 and wherein said albumin variant polypeptide specifically binds to FcRn at a pH of about 5.5.

34. A nucleic acid molecule encoding the albumin variant polypeptide of any one of claims 1-33.

35. The nucleic acid molecule of claim 34, wherein said nucleic acid molecule comprises deoxyribonucleotides.

36. The nucleic acid molecule of claim 34, wherein said nucleic acid molecule comprises ribonucleotides.

37. A vector operable to express said albumin variant polypeptide encoded by said nucleic acid molecule of any one of claims 34-46.

38. The vector of claim 37, wherein said vector comprises bacterial, bacteriophage, fungal, viral, insect, or mammalian expression control sequences.

39. A cell comprising the nucleic acid molecule of any one of claims 34-46, wherein said albumin variant polypeptide is expressed from said vector.

40. A pharmaceutical composition, comprising the albumin variant polypeptide of any one of claims 1-33.

41. A medicament, comprising the albumin variant polypeptide of any one of claims 1-33.

42. A pharmaceutical composition, comprising the nucleic acid molecule of any one of claims 34-38.

43. A medicament, comprising the nucleic acid molecule of any one of claims 34-38.

44. Use of an albumin variant polypeptide of any one of claims 1-33 in therapy.

45. A method of treating a disease, comprising administering an effective amount of a composition comprising an albumin variant polypeptide of any one of claims 1-33 to a patient in need thereof.

46. A method of manufacturing an albumin variant polypeptide of any one of claims 1-33, comprising transferring a nucleic acid molecule operable to express said albumin variant polypeptide into an expression system and expressing said albumin variant polypeptide from said nucleic acid molecule.

47. The method of claim 46, further comprising recovering said albumin variant from said expression system.

48. A method of manufacturing the albumin variant polypeptide of any one of claims 1-33, comprising synthesizing said polypeptide in an in vitro synthesis reaction.

49. The method of claim 48, wherein said in vitro synthesis reaction is selected from the group consisting of cell-free protein synthesis, liquid phase protein synthesis, and solid phase protein synthesis.
